SEQUENCE LISTING (38 sequences)

SEQ ID NO: 1 - DNA, 141 bp, synthetic construct
  source 1..141: mol_type = other DNA; note = 5' ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 166 bp, synthetic construct
  source 1..166: mol_type = other DNA; note = 3' ITR
tcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 4200 bp, synthetic construct
  source 1..4200: mol_type = other DNA; note = hSARE-ArcMin-BDNF-short 3' UTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataagcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 4098 bp, synthetic construct
  source 1..4098: mol_type = other DNA; note = hSARE-ArcMin-BDNF-bGHpA-hAlb
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagggtaccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 4630 bp, synthetic construct
  source 1..4630: mol_type = other DNA; note = hSARE-ArcMin-BDNF-long 3' UTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcagggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 595 bp, synthetic construct
  source 1..595: mol_type = other DNA; note = hSARE-ArcMin
gggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacag

SEQ ID NO: 7 - DNA, 744 bp, synthetic construct
  source 1..744: mol_type = other DNA; note = BDNF
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 8 - DNA, 327 bp, synthetic construct
  source 1..327: mol_type = other DNA; note = BDNF short 3' UTR
tggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataa

SEQ ID NO: 9 - DNA, 225 bp, synthetic construct
  source 1..225: mol_type = other DNA; note = bGH poly (A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - DNA, 2926 bp, synthetic construct
  source 1..2926: mol_type = other DNA; note = BDNF long 3' UTR
tggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaa

SEQ ID NO: 11 - DNA, 111 bp, synthetic construct
  misc_feature 40..49: note = cAMP response element
  misc_feature 53..63: note = MEF2 binding site
  misc_feature 69..88: note = Serum Response Element
  source 1..111: mol_type = other DNA; note = hSARE
gggtggtgcagagtctgcatgcgtgaggagctcctgggcgcgtcacagccgcgctattctcagcgtctctccttttatggctccggaagtgagctggggttgctggcagcc

SEQ ID NO: 12 - DNA, 484 bp, synthetic construct
  source 1..484: mol_type = other DNA; note = hArcMin
gtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacag

SEQ ID NO: 13 - DNA, 2165 bp, synthetic construct
  source 1..2165: mol_type = other DNA; note = hAlbumin stuffer sequence
gcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgc

SEQ ID NO: 14 - DNA, 12 bp, synthetic construct
  source 1..12: mol_type = other DNA; note = Kozak sequence
acccggagcagc

SEQ ID NO: 15 - skipped

SEQ ID NO: 16 - DNA, 1039 bp, synthetic construct
  source 1..1039: mol_type = other DNA; note = E-SARE-ArcMin sequence
gggtggtgcagagtctgcatgcgtgaggagctcctgggcgcgtcacagccgcgctattctcagcgtctctccttttatggctccggaagtgagctggggttgctggcagccgggtggtgcagagtctgcatgcgtgaggagctcctgggcgcgtcacagccgcgctattctcagcgtctctccttttatggctccggaagtgagctggggttgctggcagccgggtggtgcagagtctgcatgcgtgaggagctcctgggcgcgtcacagccgcgctattctcagcgtctctccttttatggctccggaagtgagctggggttgctggcagccgggtggtgcagagtctgcatgcgtgaggagctcctgggcgcgtcacagccgcgctattctcagcgtctctccttttatggctccggaagtgagctggggttgctggcagccgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacag

SEQ ID NO: 17 - DNA, 278 bp, synthetic construct
  source 1..278: mol_type = other DNA; note = promoter sequence
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 18 - DNA, 240 bp, synthetic construct
  source 1..240: mol_type = other DNA; note = promoter sequence
cggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggccgagcgggggaccgggaagc

SEQ ID NO: 19 - DNA, 379 bp, synthetic construct
  source 1..379: mol_type = other DNA; note = promoter sequence
attcctgctgggaaaagcaagtggaggtgctccttgaagaaacagggggatcccaccgatctcaggggttctgttctggcctgcggccctggatcgtccagcctgggtcggggtggggagcagacctcgcccttatcggctggggctgagggtgagggtcccgtttccccaaaggcctagcctggggttccagccacaagccctaccgggcagcgcccggccccgcccctccaggcctggcactcgtcctcaaccaagatggcgcggatggcttcaggcgcatcacgacaccggcgcgtcacgcgacccgccctacgggcacctcccgcgcttttcttagcgccgcagacggtggtcgagcgggggaccgggaagctta

SEQ ID NO: 20 - DNA, 146 bp, synthetic construct
  source 1..146: mol_type = other DNA; note = promoter sequence
atgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaa

SEQ ID NO: 21 - DNA, 301 bp, synthetic construct
  source 1..301: mol_type = other DNA; note = enhancer sequence
tacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 22 - DNA, 122 bp, synthetic construct
  source 1..122: mol_type = other DNA; note = poly A signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 23 - DNA, 56 bp, synthetic construct
  source 1..56: mol_type = other DNA; note = poly A signal
aataaaggaaatttattttcattgcaatagtgtgttggaattttttgtgtctctca

SEQ ID NO: 24 - DNA, 97 bp, synthetic construct
  source 1..97: mol_type = other DNA; note = intronic sequence
gtaagtttagtctttttgtcttttatttcaggtcccggatccggtggtggtgcaaatcaaagaactgctcctcagtggatgttgcctttacttctag

SEQ ID NO: 25 - DNA, 133 bp, synthetic construct
  source 1..133: mol_type = other DNA; note = intronic sequence
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 26 - DNA, 4008 bp, synthetic construct
  source 1..4008: mol_type = other DNA; note = C-mScar-shortUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataagcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 4443 bp, synthetic construct
  source 1..4443: mol_type = other DNA; note = C-mScar-longUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 3906 bp, synthetic construct
  source 1..3906: mol_type = other DNA; note = C-mScar-bGH
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtagggtaccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 4140 bp, synthetic construct
  source 1..4140: mol_type = other DNA; note = C-dmScar-shortUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataagcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 4575 bp, synthetic construct
  source 1..4575: mol_type = other DNA; note = C-dmScar-longUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 4038 bp, synthetic construct
  source 1..4038: mol_type = other DNA; note = C-dmScar-bGH
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtaccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 4287 bp, synthetic construct
  source 1..4287: mol_type = other DNA; note = AD-dmScar-shortUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataagcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 4722 bp, synthetic construct
  source 1..4722: mol_type = other DNA; note = AD-dmScar-longUTR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtacctggatttatgttgtatagattagattatattgagacaaaaattatctatttgtatatatacataacagggtaaattattcagttaagaaaaaaataattttatgaactgcatgtataaatgaagtttatacagtacagtggttctacaatctatttattggacatgtccatgaccagaagggaaacagtcatttgcgcacaacttaaaaagtctgcattacattccttgataatgttgtggtttgttgccgttgccaagaactgaaaacataaaaagttaaaaaaaataataaattgcatgctgctttaattgtgaattgataataaactgtcctctttcagaaaacagaaaaaaacacacacacacacaacaaaaatttgaaccaaaacattccgtttacattttagacagtaagtatcttcgttcttgttagtactatatctgttttactgcttttaacttctgatagcgttggaattaaaacaatgtcaaggtgctgttgtcattgctttactggcttaggggatgggggatggggggtatatttttgtttgttttgtgtttttttttcgtttgtttgttttgttttttagttcccacagggagtagagatggggaaagaattcctacaatatatattctggctgataaaagatacatttgtatgttgtgaagatgtttgcaatatcgatcagatgactagaaagtgaataaaaattaaggcaactgaacaaaaaaatgctcacactccacatcccgtgatgcacctcccaggccccgctcattctttgggcgttggtcagagtaagctgcttttgacggaaggacctatgtttgctcagaacacattctttccccccctccccctctggtctcctctttgttttgttttaaggaagaaaaatcagttgcgcgttctgaaatattttaccactgctgtgaacaagtgaacacattgtgtcacatcatgacactcgtataagcatggagaacagtgatttttttttagaacagaaaacaacaaaaaataaccccaaaatgaagattattttttatgaggagtgaacatttgggtaaatcatggctaagcttaaaaaaaactcatggtgaggcttaacaatgtcttgtaagcaaaaggtagagccctgtatcaacccagaaacacctagatcagaacaggaatccacattgccagtgacatgagactgaacagccaaatggaggctatgtggagttggcattgcatttaccggcagtgcgggaggaatttctgagtggccatcccaaggtctaggtggaggtggggcatggtatttgagacattccaaaacgaaggcctctgaaggacccttcagaggtggctctggaatgacatgtgtcaagctgcttggacctcgtgctttaagtgcctacattatctaactgtgctcaagaggttctcgactggaggaccacactcaagccgacttatgcccaccatcccacctctggataattttgcataaaattggattagcctggagcaggttgggagccaaatgtggcatttgtgatcatgagattgatgcaatgagatagaagatgtttgctacctgaacacttattgctttgaaactagacttgaggaaaccagggtttatcttttgagaacttttggtaagggaaaagggaacaggaaaagaaaccccaaactcaggccgaatgatcaaggggacccataggaaatcttgtccagagacaagacttcgggaaggtgtctggacattcagaacaccaagacttgaaggtgccttgctcaatggaagaggccaggacagagctgacaaaattttgctccccagtgaaggccacagcaaccttctgcccatcctgtctgttcatggagagggtccctgcctcacctctgccattttgggttaggagaagtcaagttgggagcctgaaatagtggttcttggaaaaatggatccccagtgaaaactagagctctaagcccattcagcccatttcacacctgaaaatgttagtgatcaccacttggaccagcatccttaagtatcagaaagccccaagcaattgctgcatcttagtagggtgagggataagcaaaagaggatgttcaccataacccaggaatgaagataccatcagcaaagaatttcaatttgttcagtctttcatttagagctagtctttcacagtaccatctgaatacctctttgaaagaaggaagactttacgtagtgtagatttgttttgtgttgtttgaaaatattatctttgtaattatttttaatatgtaaggaatgcttggaatatctgctatatgtcaactttatgcagcttccttttgagggacaaatttaaaacaaacaaccccccatcacaaacttaaaggattgcaagggccagatctgttaagtggtttcataggagacacatccagcaattgtgtggtcagtggctcttttacccaataagatacatcacagtcacatgcttgatggtttatgttgacctaagatttattttgttaaaatctctctctgttgtgttcgttcttgttctgttttgttttgttttttaaagtcttgctgtggtctctttgtggcagaagtgtttcatgcatggcagcaggcctgttgcttttttatggcgattcccattgaaaatgtaagtaaatgtctgtggccttgttctctctatggtaaagatattattcaccatgtaaaacaaaaaacaatatttattgtattttagtatatttatataattatgttattgaaaaaaattggcattaaaacttaaccgcatcagaacctattgtaaatacaagttctatttaagtgtactaattaacatataatatatgttttaaatatagaatttttaatgtttttaaatatattttcaaagtacataaaactcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 4185 bp, synthetic construct
  source 1..4185: mol_type = other DNA; note = AD-dmScar-bGH Construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacaggcctcctaggcggaccgcttgcatgcacgcgtgggtggtgcagagtctgcatgcgtgaggagctcctgggcgcgtcacagccgcgctattctcagcgtctctccttttatggctccggaagtgagctggggttgctggcagccgtccctcggtccccgcaggctgaatgagccccgccgtccggggtgggaggtacggagcggagggattagtcatcctggccccgccatgtgtgcagggggtgggacctcccgtcccttgccgcccgggagccgagtgggcgcagggcggggcctgcaggggcgcgggcggggcgctggcggggagcctgcggctgggccaatgagaaaccgggctcggcgagccggccgcccacgggcctcgctggctgcataaagagccggcggccaggactcagcgcagagctcgggcgcggcgtcctccctccgcagcagccgagccggacctgcctccccgggcgtgctccgccggccccgccgccggcccgcagcgacagacaggcgctccccgcagctccgcacgggacccaggccgccggaccccagcgccggaccaccctctgtccgccccgaggagtttgccgcctgccggagcacctgcgcacagaccggtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtagggtaccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgctcgagccatgggcgcgccatcgatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - DNA, 831 bp, synthetic construct
  source 1..831: mol_type = other DNA; note = dmScarlet (mScarlet-I with Cterminal PEST degron)
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagaagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtag

SEQ ID NO: 36 - DNA, 696 bp, synthetic construct
  source 1..696: mol_type = other DNA; note = mScarlet-I
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 37 - DNA, 135 bp, synthetic construct
  source 1..135: mol_type = other DNA; note = C-terminal PEST degron
aagcttccgcggagccatggcttcccgccggcggtggcggcgcaggatgatggcacgctgcccatgtcttgtgcccaggagagcgggatggaccgtcaccctgcagcctgtgcttctgctaggatcaatgtgtag

SEQ ID NO: 38 - DNA, 448 bp, synthetic construct
  source 1..448: mol_type = other DNA; note = hSyn
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

(19) United States
(12) Patent Application Publication
(10) Pub. No.: US 2024/0392316 A1
(43) Pub. Date: Nov. 28, 2024

(54) ADENO-ASSOCIATED VIRUS VECTORS FOR TREATMENT OF RETT SYNDROME

(71) Applicant: Sarepta Therapeutics, Inc., Cambridge, MA (US)

(72) Inventor: Colin O'Banion, Cambridge, MA (US)

(73) Assignee: Sarepta Therapeutics, Inc., Cambridge, MA (US)

(21) Appl. No.: 18/680,380

(22) Filed: May 31, 2024

Related U.S. Application Data
(62) Division of application No. 17/406,723, filed on Aug. 19, 2021, now Pat. No. 12,104,163.
(60) Provisional application No. 63/067,668, filed on Aug. 19, 2020.

(51) Int. Cl. C12N 15/86; A61K 38/18; A61K 48/00; A61P 25/28; C07K 14/475; C07K 14/61

(52) U.S. Cl. CPC C12N 15/86; A61K 38/185; A61K 48/0058; A61K 48/0066; A61P 25/28; C07K 14/475; C07K 14/61; C12N 2750/14143; C12N 2750/14171; C12N 2830/008; C12N 2830/15; C12N 2830/38; C12N 2830/50

(57) ABSTRACT

The disclosure provides nucleic acids (comprising AAV expression cassettes), AAV vectors, and compositions for use in methods for treating and/or delaying the onset of diseases associated with mutations in the mecp2 gene, such as Rett Syndrome. Also, provided herein are methods for treating and/or delaying the onset of brain-derived neurotrophic factor (BDNF)-associated diseases.

### CROSS-REFERENCE TO RELATED APPLICATIONS

This application is a divisional of U.S. application Ser. No. 17/406,723, filed Aug. 19, 2021, which claims priority to U.S. Provisional Application No. 63/067,668, filed Aug. 19, 2020, the contents of each of which are incorporated herein by reference in their entirety.

### FIELD

The instant disclosure relates to the fields of molecular biology and gene therapy. More specifically, disclosure relates to compositions and methods for producing recombinant viral vectors.

### INCORPORATION OF THE SEQUENCE LISTING

The contents of the text file submitted electronically herewith are incorporated herein by reference in their entirety: A computer readable format copy of the Sequence Listing (Name: 4140_0680003_Sequence_Listing_ST26.xml; Size: 105,115 bytes; and Date of Creation: Jul. 10, 2024).

### BACKGROUND

Rett Syndrome is a genetic neurological disorder manifesting at about 6 months to 18 months of age primarily in girls and women. Affecting about 1 in 8,500 females, Rett Syndrome is a rare disease. Patients with Rett Syndrome often exhibit a wide range of symptoms including language disability, reduced coordination, microcephaly, repetitive movements, seizures, scoliosis, and cognitive disability. While the life expectancy for women with Rett Syndrome is in the mid-40s, males with Rett Syndrome often die in infancy.

Rett Syndrome is caused by mutations in the mecp2 gene, and is correlated with defects in neuronal function. Many cases of Rett syndrome result from new mutations in the mecp2 gene. However, in a small number of cases, mecp2 mutations show an X-linked, dominant pattern of inheritance. Mutations in mecp2 also cause other diseases such as MECP2 duplication syndrome and PPM-X syndrome, and may be associated with Autism Spectrum disorder.

There is no known treatment for Rett Syndrome, or other mecp2-associated diseases. Rett Syndrome symptoms are typically managed using anticonvulsants, special education, physiotherapy, and braces. Gene therapy is a potentially useful method to treat Rett Syndrome. However, gene therapy targeted to the nervous system often faces several technical hurdles, including identifying the right neuronal target genes that would alleviate symptoms upon expression, promoting high levels of expression of these genes in a regulated manner, and delivering the gene therapy constructs in a targeted manner to neurons. Accordingly, there is an unmet need for compositions and methods that treat Rett Syndrome, and other mecp2-associated diseases.

### SUMMARY

The disclosure provides nucleic acids comprising an adeno-associated virus (AAV) expression cassette, wherein the AAV expression cassette comprises, from 5′ to 3′: a 5′ inverted terminal repeat (ITR); a synthetic, activity-dependent promoter; a Rett Syndrome-associated gene; and a 3′ ITR. In some embodiments, the promoter drives expression of the Rett-syndrome-associated gene. In some embodiments, the promoter is a MECP2-independent promoter. In some embodiments, the promoter comprises a nucleic acid sequence derived from a promoter of a neuronal immediate early gene. In some embodiments, the neuronal immediate early gene is selected from the group consisting of the Arc gene, the c-fos gene, and the egr-1 gene. In some embodiments, the promoter comprises a minimal Arc gene promoter (ArcMin).

In some embodiments, the ArcMin is a human ArcMin (hArcMin). In some embodiments, the hArcMin comprises a nucleic acid sequence of SEQ ID NO: 12, or a sequence at least 90% identical thereto. In some embodiments, the promoter comprises a cyclic AMP response element (CRE), a serum response element (SRE), a synaptic activity response element (SARE), a MEF2 response element, or a combination thereof. In some embodiments, the promoter comprises a synaptic activity response element (SARE). In some embodiments, the synaptic activity response element (SARE) is a human synaptic activity response element (hSARE). In some embodiments, the hSARE comprises a nucleic acid sequence of SEQ ID NO: 11, or a sequence at least 90% identical thereto. In some embodiments, the promoter comprises a human ArcMin (hArcMin) and at least one hSARE. In some embodiments, the promoter comprises hArcMin and one hSARE. In some embodiments, the promoter comprises a nucleic acid sequence of SEQ ID NO: 6, or a sequence at least 90% identical thereto. In some embodiments, the promoter comprises hArcMin and five hSAREs. In some embodiments, the promoter comprises a nucleic acid sequence of SEQ ID NO: 16, or a sequence at least 90% identical thereto.

In some embodiments, the promoter binds to a neuronal activity dependent transcription factor. In some embodiments, the neuronal activity dependent transcription factor is cAMP-responsive element binding protein (CREB), myocyte enhancer factor 2 (MEF2), serum response factor (SRF), or Elk-1. In some embodiments, the Rett-syndrome-associated gene encodes brain-derived neurotrophic factor (BDNF), insulin-like growth factor 1 (IGF1), methyl-CpG-binding protein 2 (MECP2), Huntingtin protein, Huntington-associated protein 1, Orthodenticle homeobox 2 (OTX-2), FXYD Domain Containing Ion Transport Regulator 1 (FXYD1), Neurexin-2-alpha (NRXN2), or Protein Kinase C Gamma (PRKCG). In some embodiments, the Rett-syndrome-associated gene encodes brain-derived neurotrophic factor (BDNF). In some embodiments, the BDNF is a human BDNF. In some embodiments, the BDNF is encoded by a nucleic acid sequence of SEQ ID NO: 7, or a sequence at least 90% identical thereto.

In some embodiments, at least one of the 5′ ITR and the 3′ ITR is about 110 to about 160 nucleotides in length. In some embodiments, the 5′ ITR is the same length as the 3′ ITR. In some embodiments, the 5′ ITR and the 3′ ITR have different lengths. In some embodiments, at least one of the 5′ ITR and the 3′ ITR is isolated or derived from the genome of AAV1, AAV2, AAV3, AAV4, AAV5, AAV6, AAV7, AAV8, AAV9, AAVIO, AAV11, AAV12, AAVrh8, AAVrh10, AAVrh32.33, AAVrh74, Avian AAV or Bovine AAV. In some embodiments, the 5′ ITR comprises the sequence of SEQ ID NO: 1. In some embodiments, the 3′ ITR comprises the sequence of SEQ ID NO: 2.

In some embodiments, the AAV cassette comprises a brain-derived neurotrophic factor (BDNF) short 3′ UTR, or a BDNF long 3′ UTR. In some embodiments, the BDNF short 3′ UTR, or the BDNF long 3′ UTR is located between the Rett-syndrome-associated gene and the 3′ ITR. In some embodiments, the AAV cassette comprises a BDNF short 3′ UTR. In some embodiments, the BDNF short 3′ UTR comprises a nucleic acid sequence of SEQ ID NO: 8, or a sequence at least 90% identical thereto. In some embodiments, the AAV cassette comprises a BDNF long 3′ UTR. In some embodiments, the BDNF long 3′ UTR comprises a nucleic acid sequence of SEQ ID NO: 10, or a sequence at least 90% identical thereto.

In some embodiments, the AAV cassette comprises a polyadenylation signal. In some embodiments, the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH). In some embodiments, the AAV cassette comprises a bGH polyadenylation signal. In some embodiments, the bGH polyadenylation signal comprises a nucleic acid sequence of SEQ ID NO: 9, or a sequence at least 90% identical thereto.

In some embodiments, the AAV cassette comprises at least one stuffer sequence. In some embodiments, the at least one stuffer sequence comprises a nucleic acid sequence of SEQ ID NO: 13, or a sequence at least 90% identical thereto. In some embodiments, the AAV expression cassette comprises a Kozak sequence, wherein the Kozak sequence overlaps with the start codon of the Rett-syndrome-associated gene. In some embodiments, the Kozak sequence comprises a nucleic acid sequence of SEQ ID NO: 14, or a sequence at least 90% identical thereto; or a nucleic acid sequence of SEQ ID NO: 15 (acagccacc), or a sequence at least 90% identical thereto. In some embodiments, the AAV expression cassette comprises a nucleic acid sequence SEQ ID NO: 3, or a sequence at least 90% identical thereto; SEQ ID NO: 4 or a sequence at least 90% identical thereto; or SEQ ID NO: 5 or a sequence at least 90% identical thereto.

The disclosure provides plasmids comprising any one of the nucleic acids disclosed herein. The disclosure also provides cells comprising any one of the nucleic acids disclosed herein or any one of the plasmids disclosed herein. The disclosure further provides methods of producing a recombinant AAV vector, the method comprising contacting an AAV producer cell with any one of the nucleic acids disclosed herein, or any one of the plasmids disclosed herein. The disclosure provides recombinant AAV vectors produced by any one of the methods of producing a recombinant AAV vector disclosed herein. In some embodiments, the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vector is a single-stranded AAV (ssAAV). In some embodiments, the recombinant AAV vector is a self-complementary AAV (scAAV). In some embodiments, the AAV vector comprises a capsid protein of AAV1, AAV2, AAV3, AAV4, AAV5, AAV6, AAV7, AAV8, AAV9, AAVIO, AAV11, AAV12, AAVrh8, AAVrh10, AAVrh32.33, AAVrh74, Avian AAV or Bovine AAV. In some embodiments, the AAV vector comprises a capsid protein with one or more substitutions or mutations, as compared to a wildtype AAV capsid protein.

The disclosure provides compositions, comprising: (a) any one of the nucleic acids disclosed herein, any one of the plasmids disclosed herein, any one of the cells disclosed herein, or any one of the recombinant AAV vectors disclosed herein; and (b) a pharmaceutically acceptable carrier. The disclosure provides methods for expressing a Rett-syndrome-associated gene in a subject in need thereof, comprising administering to the subject a therapeutically effective amount of any one of the nucleic acids disclosed herein, any one of the plasmids disclosed herein, any one of the cells disclosed herein, or any one of the recombinant AAV vectors disclosed herein, or any one of the compositions disclosed herein. In some embodiments, the subject has Rett syndrome.

The disclosure provides methods for treating or delaying the onset of Rett syndrome in a subject, comprising administering to the subject a therapeutically effective amount of any one of the nucleic acids disclosed herein, any one of the plasmids disclosed herein, any one of the cells disclosed herein, or any one of the recombinant AAV vectors disclosed herein, or any one of the compositions disclosed herein. The disclosure provides methods for expressing brain-derived neurotrophic factor (BDNF) in a subject in need thereof, comprising administering to the subject a therapeutically effective amount of any one of the nucleic acids disclosed herein, any one of the plasmids disclosed herein, any one of the cells disclosed herein, or any one of the recombinant AAV vectors disclosed herein, or any one of the compositions disclosed herein.

In some embodiments, the subject has a cognitive disorder, or a stress-related disorder. In some embodiments, the subject has depression, obsessive-compulsive disorder, Alzheimer's disease, Huntington's disease, and dementia, anorexia nervosa and bulimia nervosa, schizophrenia, epilepsy, post-traumatic stress disorder, obesity, Rett syndrome, or post-chemotherapy cognitive impairment.

The disclosure provides methods for treating or delaying the onset of a BDNF-associated disease in a subject, comprising administering to the subject a therapeutically effective amount of any one of the nucleic acids disclosed herein, any one of the plasmids disclosed herein, any one of the cells disclosed herein, or any one of the recombinant AAV vectors disclosed herein, or any one of the compositions disclosed herein. In some embodiments, the BDNF-associated disease is a cognitive disorder, and/or a stress-related disorder. In some embodiments, the BDNF-associated disease is depression, obsessive-compulsive disorder, Alzheimer's disease, Huntington's disease, and dementia, anorexia nervosa, bulimia nervosa, schizophrenia, epilepsy, post-traumatic stress disorder, bipolar disease, Rett syndrome, major depressive disorder, or post-chemotherapy cognitive impairment.

The disclosure provides methods for treating or delaying the onset of a MECP2-associated disease in a subject, comprising administering to the subject a therapeutically effective amount of any one of the nucleic acids disclosed herein, any one of the plasmids disclosed herein, any one of the cells disclosed herein, or any one of the recombinant AAV vectors disclosed herein, or any one of the compositions disclosed herein. In some embodiments, the MECP2-associated disease is MECP2 duplication syndrome, MECP2-related severe neonatal encephalopathy, PPM-X syndrome, or Rett syndrome. In some embodiments, the subject is a human subject. In some embodiments, the nucleic acid, the plasmid, the cell, the recombinant AAV vector, or composition is administered by injection into the central nervous system. In some embodiments, the Rett Syndrome-associated gene is expressed in the neurons of the subject. In some embodiments, the neurons are active neurons.

These and other embodiments are addressed in more detail in the detailed description set forth below.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 shows an AAV expression cassette, comprising the hSARE-hArcMin promoter (SEQ ID NO: 6), BDNF gene (SEQ ID NO: 7), the BDNF short 3′ UTR (SEQ ID NO: 8) and a stuffer sequence (SEQ ID NO: 13), all of which are flanked by a 5′ ITR (SEQ ID NO: 1) and a 3′ ITR (SEQ ID NO: 2).

FIG. 2 shows an AAV expression cassette, comprising the hSARE-hArcMin promoter (SEQ ID NO: 6), BDNF gene (SEQ ID NO: 7), bGH polyA signal (SEQ ID NO: 9) and a stuffer sequence (SEQ ID NO: 13), all of which are flanked by a 5′ ITR (SEQ ID NO: 1) and a 3′ ITR (SEQ ID NO: 2).

FIG. 3 shows an AAV expression cassette, comprising the hSARE-hArcMin promoter (SEQ ID NO: 6), BDNF gene (SEQ ID NO: 7), and BDNF long 3′ UTR (SEQ ID NO: 10), all of which are flanked by a 5′ ITR (SEQ ID NO: 1) and a 3′ ITR (SEQ ID NO: 2).

FIG. 4 shows the schematic representations of AAV expression cassettes for reporter gene expression comprising a constitutive (“C”) promoter, hSyn or an activity-dependent (“AD”) promoter, hSARE-hArcMin. The elements of the expression cassettes are also listed in Table 3.

FIG. 5A is a bar graph showing the reporter protein fluorescence in cells that are not treated with tetrodotoxin (TTX), relative to the fluorescence of cells that are treated with 2 μM TTX. FIG. 5B is a bar graph showing the reporter protein fluorescence in cells that are treated with 150 mM KCl, relative to the fluorescence of cells that are treated with 2 μM TTX. FIG. 5C is a bar graph showing the reporter protein fluorescence in cells that are treated with 30 uM bicuculine (BIC), relative to the fluorescence of cells that are treated with 2 μM TTX. Each of the bars shows the relative fluorescence of cells transduced with AAV vectors comprising the indicated AAV expression cassettes at the multiplicity of infection (MOI) indicated on the X axis.

FIG. 6A is a graph showing the relative reporter gene expression in cells transduced with AAV vectors comprising the indicated AAV expression cassettes, which were treated with 30 μM of BIC and further either treated with 2 μM TTX, or left untreated with TTX, as indicated. FIG. 6B is a bar graph showing the relative reporter gene expression in cells transduced with AAV vectors comprising the activity-dependent (AD) AAV expression cassettes, which were treated with 30 μM of BIC and further either treated with 2 μM TTX, or left untreated with TTX, as indicated.

FIG. 7 depicts microscopy images showing mScarlet fluorescence (top images) and staining with the neuronal marker, anti-beta III Tubulin antibody (bottom images) of neurons transduced with AAV vectors comprising the indicated constitutive (C) AAV expression cassettes.

FIG. 8 depicts microscopy images showing: Hoescht DNA staining, dmScarlet fluorescence, anti-beta III Tubulin antibody staining and overlay images, of neurons transduced with AAV vectors comprising the “AD-dmScar-bGH” AAV expression cassette. The top panel shows images of cells treated with 2 μM TTX and 30 μM of BIC, and the bottom panel shows images of cells which were left untreated with TTX but treated with 30 μM of BIC.

FIG. 9A shows an AAV expression cassette, comprising the nucleic acid sequence of SEQ ID NO: 26, and comprising the following elements: hSyn promoter (SEQ ID NO: 38), mScarlet gene (SEQ ID NO: 36), the BDNF short 3′ UTR (SEQ ID NO: 8) and a stuffer sequence (SEQ ID NO: 13), all of which are flanked by a 5′ ITR (SEQ ID NO: 1) and a 3′ ITR (SEQ ID NO: 2). FIG. 9B shows an AAV expression cassette, comprising the nucleic acid sequence of SEQ ID NO: 27, and comprising the following elements: the hSyn promoter (SEQ ID NO: 38), mScarlet gene (SEQ ID NO: 36), and the BDNF long 3′ UTR (SEQ ID NO: 10), all of which are flanked by a 5′ ITR (SEQ ID NO: 1) and a 3′ ITR (SEQ ID NO: 2). FIG. 9C shows an AAV expression cassette, comprising the nucleic acid sequence of SEQ ID NO: 28, and comprising the following elements: the hSyn promoter (SEQ ID NO: 38), mScarlet gene (SEQ ID NO: 36), the bGH poly A signal (SEQ ID NO: 9) and a stuffer sequence (SEQ ID NO: 13), all of which are flanked by a 5′ ITR (SEQ ID NO: 1) and a 3′ ITR (SEQ ID NO: 2).

FIG. 10A shows an AAV expression cassette, comprising the nucleic acid sequence of SEQ ID NO: 29, and comprising the following elements: the hSyn promoter (SEQ ID NO: 38), dmScarlet gene (SEQ ID NO: 35), the BDNF short 3′ UTR (SEQ ID NO: 8) and a stuffer sequence (SEQ ID NO: 13), all of which are flanked by a 5′ ITR (SEQ ID NO: 1) and a 3′ ITR (SEQ ID NO: 2). FIG. 10B shows an AAV expression cassette, comprising the nucleic acid sequence of SEQ ID NO: 30, and comprising the following elements: the hSyn promoter (SEQ ID NO: 38), dmScarlet gene (SEQ ID NO: 35), and the BDNF long 3′ UTR (SEQ ID NO: 10), all of which are flanked by a 5′ ITR (SEQ ID NO: 1) and a 3′ ITR (SEQ ID NO: 2). FIG. 10C shows an AAV expression cassette, comprising the nucleic acid sequence of SEQ ID NO: 31, and comprising the following elements: the hSyn promoter (SEQ ID NO: 38), dmScarlet gene (SEQ ID NO: 35), the bGH poly A signal (SEQ ID NO: 9) and a stuffer sequence (SEQ ID NO: 13), all of which are flanked by a 5′ ITR (SEQ ID NO: 1) and a 3′ ITR (SEQ ID NO: 2).

FIG. 11A shows an AAV expression cassette, comprising the nucleic acid sequence of SEQ ID NO: 32, and comprising the following elements: the hSARE-hArcMin promoter (SEQ ID NO: 6), dmScarlet gene (SEQ ID NO: 35), the BDNF short 3′ UTR (SEQ ID NO: 8) and a stuffer sequence (SEQ ID NO: 13), all of which are flanked by a 5′ ITR (SEQ ID NO: 1) and a 3′ ITR (SEQ ID NO: 2). FIG. 11B shows an AAV expression cassette, comprising the nucleic acid sequence of SEQ ID NO: 33, and comprising the following elements: the hSARE-hArcMin promoter (SEQ ID NO: 6), dmScarlet gene (SEQ ID NO: 35), and the BDNF long 3′ UTR (SEQ ID NO: 10), all of which are flanked by a 5′ ITR (SEQ ID NO: 1) and a 3′ ITR (SEQ ID NO: 2). FIG. 11C shows an AAV expression cassette, comprising the nucleic acid sequence of SEQ ID NO: 34, and comprising the following elements: the hSARE-hArcMin promoter (SEQ ID NO: 6), dmScarlet gene (SEQ ID NO: 35), the bGH polyA signal (SEQ ID NO: 9) and a stuffer sequence (SEQ ID NO: 13), all of which are flanked by a 5′ ITR (SEQ ID NO: 1) and a 3′ ITR (SEQ ID NO: 2).

FIG. 12 depicts microscopy images showing: Hoescht DNA staining, dmScarlet fluorescence, anti-beta III Tubulin antibody staining and overlay images, of neurons transduced with AAV vectors comprising the “AD-dmScar-longUTR” AAV expression cassette. The top panel shows images of cells treated with 2 μM TTX and 30 μM of BIC, and the bottom panel shows images of cells which were left untreated with TTX but treated with 30 μM of BIC.

### DETAILED DESCRIPTION

Rett Syndrome is a rare genetic neurological disorder that occurs primarily in girls. The symptoms of the syndrome, which are apparent after 6 months to 18 months of age, may include one or more of the following: problems with language, problems with coordination, repetitive movements, slower growth, problems walking, smaller head size, seizures, scoliosis, cognitive disability, and sleeping problems. Patients with Rett Syndrome may exhibit any combination of these symptoms, with varying degrees of severity of each symptom.

Rett Syndrome is caused by a genetic mutation in the mecp2 gene, which encodes the methyl CpG binding protein 2 (MECP2) protein. See UniProt Accession No. P51608, incorporated herein by reference in its entirety. More than 620 mutations in the mecp2 gene, many of which are single base pair insertions or deletions, have been identified in females with Rett syndrome. MECP2 contributes to the normal functioning of nerve cells and is present in high levels in mature nerve cells. The methyl-CpG binding (MBD) of MECP2 domain can recognize DNA regions with 5-methyl cytosine modification. Mutations in the mecp2 gene can also cause other diseases such as MECP2 duplication syndrome, MECP2-related severe neonatal encephalopathy, and PPM-X syndrome. There are no approved therapies to treat any of the diseases associated with mutations in the mecp2 gene.

The disclosure provides nucleic acids (comprising AAV expression cassettes), AAV vectors, and compositions for use in methods for treating and/or delaying the onset of diseases associated with mutations in the mecp2 gene, such as Rett Syndrome. Also, provided herein are methods for treating and/or delaying the onset of brain-derived neurotrophic factor (BDNF)-associated diseases.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

The term “wild type” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The term “mutant protein” is a term of the art understood by skilled persons and refers to a protein that is distinguished from the wild type form of the protein on the basis of the presence of amino acid modifications, such as, for example, amino acid substitutions, insertions and/or deletions. The term “mutant gene” is a term of the art understood by skilled persons and refers to a gene that is distinguished from the wild type form of the gene on the basis of the presence of nucleic acid modifications, such as, for example, nucleic acid substitutions, insertions and/or deletions. In some embodiments, the mutant gene encodes a mutant protein.

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or to increase the stability and half-life of such molecules in physiological environments. Nucleic acids of the disclosure may be linear, or may be circular (e.g., a plasmid).

As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “AAV expression cassette” is a nucleic acid that gets packaged into a recombinant AAV vector, and comprises a sequence encoding one or more transgenes. When the AAV vector is contacted with a target cell, the transgenes are expressed by the target cell.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors of the disclosure include adenovirus vectors, adeno-associated virus vectors (AAVs), lentivirus vectors, and retrovirus vectors.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11,AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80,AAV PHP.B, and any other AAV now known or later discovered. See, e.g., Table 1.

TABLE 1
Adeno-Associated Virus Serotypes

| | GenBank | | GenBank | | GenBank |
|---|---|---|---|---|---|
| | Accession | | Accession | | Accession |
| | Number | | Number | | Number |
| Complete | | Clade C | | Rh57 | AY530569 |
| Genomes | | | | | |
| Adeno- | NC_002077, | Hu9 | AY530629 | Rh50 | AY530563 |
| associated | AF063497 | | | | |
| virus 1 | | | | | |
| Adeno- | NC_001401 | Hu10 | AY530576 | Rh49 | AY530562 |
| associated | | | | | |
| virus 2 | | | | | |
| Adeno- | NC_001729 | Hu11 | AY530577 | Hu39 | AY530601 |
| associated | | | | | |
| virus 3 | | | | | |
| Adeno- | NC_001863 | Hu53 | AY530615 | Rh58 | AY530570 |
| associated | | | | | |
| virus | | | | | |
| 3B | | | | | |
| Adeno- | NC_001829 | Hu55 | AY530617 | Rh61 | AY530572 |
| associated | | | | | |
| virus 4 | | | | | |
| Adeno- | Y18065, | Hu54 | AY530616 | Rh52 | AY530565 |
| associated | AF085716 | | | | |
| virus 5 | | | | | |
| Adeno- | NC_001862, | Hu7 | AY530628 | Rh53 | AY530566 |
| associated | AAB95450.1 | | | | |
| virus 6 | | | | | |
| Avian AAV | AY186198, | Hu18 | AY530583 | Rh51 | AY530564 |
| ATCC | AY629583, | | | | |
| VR-865 | NC_004828 | | | | |
| Avian AAV | NC_006263, | Hu15 | AY530580 | Rh64 | AY530574 |
| strain DA-1 | AY629583 | | | | |
| Bovine AAV | NC_005889, | Hu16 | AY530581 | Rh43 | AY530560 |
| | AY388617, | | | | |
| | AAR26465 | | | | |
| AAV11 | AAT46339, | Hu25 | AY530591 | AAV8 | AF513852 |
| | AY631966 | | | | |
| AAV12 | ABI16639, | Hu60 | AY530622 | Rh8 | AY242997 |
| | DQ813647 | | | | |
| Clade A | | Ch5 | AY243021 | Rh1 | AY530556 |
| AAV1 | NC_002077, | Hu3 | AY530595 | Clade F | |
| | AF063497 | | | | |
| AAV6 | NC_001862 | Hu1 | AY530575 | Hu14 | AY530579 |
| | | | | (AAV9) | |
| Hu.48 | AY530611 | Hu4 | AY530602 | Hu31 | AY530596 |
| Hu 43 | AY530606 | Hu2 | AY530585 | Hu32 | AY530597 |
| Hu 44 | AY530607 | Hu61 | AY530623 | HSC1 | MI332400.1 |
| Hu 46 | AY530609 | Clade D | | HSC2 | MI332401.1 |
| Clade B | | Rh62 | AY530573 | HSC3 | MI332402.1 |
| Hu. 19 | AY530584 | Rh48 | AY530561 | HSC4 | MI332403.1 |
| Hu. 20 | AY530586 | Rh54 | AY530567 | HSC5 | MI332405.1 |
| Hu 23 | AY530589 | Rh55 | AY530568 | HSC6 | MI332404.1 |
| Hu22 | AY530588 | Cy2 | AY243020 | HSC7 | MI332407.1 |
| Hu24 | AY530590 | AAV7 | AF513851 | HSC8 | MI332408.1 |
| Hu21 | AY530587 | Rh35 | AY243000 | HSC9 | MI332409.1 |
| Hu27 | AY530592 | Rh37 | AY242998 | HSC11 | MI332406.1 |
| Hu28 | AY530593 | Rh36 | AY242999 | HSC12 | MI332410.1 |
| Hu 29 | AY530594 | Cy6 | AY243016 | HSC13 | MI332411.1 |
| Hu63 | AY530624 | Cy4 | AY243018 | HSC14 | MI332412.1 |
| Hu64 | AY530625 | Cy3 | AY243019 | HSC15 | MI332413.1 |
| Hu13 | AY530578 | Cy5 | AY243017 | HSC16 | MI332414.1 |
| Hu56 | AY530618 | Rh13 | AY243013 | HSC17 | MI332415.1 |
| Hu57 | AY530619 | Clade E | | Hu68 | |
| Hu49 | AY530612 | Rh38 | AY530558 | Clonal | |
| | | | | Isolate | |
| Hu58 | AY530620 | Hu66 | AY530626 | AAV5 | Y18065, |
| | | | | | AF085716 |
| Hu34 | AY530598 | Hu42 | AY530605 | AAV 3 | NC_001729 |
| Hu35 | AY530599 | Hu67 | AY530627 | AAV 3B | NC_001863 |
| AAV2 | NC_001401 | Hu40 | AY530603 | AAV4 | NC_001829 |
| Hu45 | AY530608 | Hu41 | AY530604 | Rh34 | AY243001 |
| Hu47 | AY530610 | Hu37 | AY530600 | Rh33 | AY243002 |
| Hu51 | AY530613 | Rh40 | AY530559 | Rh32 | AY243003 |
| Hu52 | AY530614 | Rh2 | AY243007 | Others | |
| Hu T41 | AY695378 | Bb1 | AY243023 | Rh74 | |
| Hu S17 | AY695376 | Bb2 | AY243022 | Bearded | |
| | | | | Dragon | |
| | | | | AAV | |
| Hu T88 | AY695375 | Rh10 | AY243015 | Snake | NC_006148.1 |
| | | | | AAV | |
| Hu T71 | AY695374 | Hu17 | AY530582 | | |
| Hu T70 | AY695373 | Hu6 | AY530621 | | |
| Hu T40 | AY695372 | Rh25 | AY530557 | | |
| Hu T32 | AY695371 | Pi2 | AY530554 | | |
| Hu T17 | AY695370 | Pi1 | AY530553 | | |
| Hu LG15 | AY695377 | Pi3 | AY530555 | | |

The terms “viral production cell”, “viral production cell line,” or “viral producer cell” refer to cells used to produce viral vectors. HEK293 and 239T cells are common viral production cell lines. Table 2, below, lists exemplary viral production cell lines for various viral vectors.

TABLE 2
Exemplary viral production cell lines

| Virus Vector | Exemplary Viral |
|---|---|
| | Production Cell Line(s) |
| Adenovirus | HEK293, 911, pTG6559, |
| | PER.C6, GH329, N52.E6, |
| | HeLa-E1, UR, VLI-293 |
| Adeno-Associated | HEK293, Sf9 |
| Virus (AAV) | |
| Retrovirus | HEK293 |
| Lentivirus | 293T |

“HEK293” refers to a cell line originally derived from human embryonic kidney cells grown in tissue culture. The HEK293 cell line grows readily in culture, and is commonly used for viral production. As used herein, “HEK293” may also refer to one or more variant HEK293 cell lines, i.e., cell lines derived from the original HEK293 cell line that additionally comprise one or more genetic alterations. Many variant HEK293 lines have been developed and optimized for one or more particular applications. For example, the 293T cell line contains the SV40 large T-antigen that allows for episomal replication of transfected plasmids containing the SV40 origin of replication, leading to increased expression of desired gene products.

“Sf9” refers to an insect cell line that is a clonal isolate derived from the parental Spodoptera frugiperda cell line IPLB-Sf-21-AE. Sf9 cells can be grown in the absence of serum and can be cultured attached or in suspension.

A “transfection reagent” means a composition that enhances the transfer of nucleic acid into cells. Some transfection reagents commonly used in the art include one or more lipids that bind to nucleic acids and to the cell surface (e.g., Lipofectamine™).

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection.

Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al, (1997) Nucleic Acids Res. 25, 3389-3402.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the term “gene therapy” refers to the process of introducing genetic material into cells to compensate for abnormal genes, or to make a therapeutic protein.

As used herein, a neuron is said to be an “active” neuron or to have “neuronal activity” when at least a part of the neuronal plasma membrane is depolarized. In some embodiments, the depolarization is caused by opening of voltage-gated Na channels. In some embodiments, the depolarization of the neuronal plasma membrane leads to the influx of Ca²⁺ ions into the cell and triggers downstream transcription of immediate early genes (IEG). In vivo, depolarization of at least a part of the plasma membrane of a post-synaptic neuron may be triggered by a signal from a pre-synaptic neuron. In vitro, depolarization may be induced by treating neurons with KCl. Depolarization may also be promoted in vitro by using small molecules (e.g. bicuculline) to inactivate signal receptors, such as γ-Aminobutyric acid type A (GABAA) receptor, which normally slows down depolarization by opening Cl⁻ or K⁺ channels.

As used herein, a neuron is said to be a “resting” neuron when there is no net flow of ions across the plasma membrane of the neuron. In some embodiments, no part of the resting neuronal plasma membrane is depolarized. In some embodiments, the resting neuron has a resting membrane potential of about −70 mV.

### AAV Expression Cassettes

The disclosure provides nucleic acid sequences comprising one or more adeno-associated virus (AAV) expression cassettes. In some embodiments, the AAV expression cassette comprises a 5′ inverted terminal repeat (ITR), a promoter, a transgene, and a 3′ ITR. In some embodiments, the transgene is a Rett Syndrome-associated gene. In some embodiments, the AAV expression cassette comprises a Kozak sequence, a polyadenylation sequence, and/or a stuffer sequence.

In some embodiments, the AAV expression cassette comprises a nucleic acid sequence of SEQ ID NO: 3, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween). In some embodiments, the AAV expression cassette comprises a nucleic acid sequence of SEQ ID NO: 4, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the AAV expression cassette comprises a nucleic acid sequence of SEQ ID NO: 5, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween).

### (i) Inverted Terminal Repeat

Inverted Terminal Repeat or ITR sequences are sequences that mediate AAV proviral integration and packaging of AAV DNA into virions. ITRs are involved in a variety of activities in the AAV life cycle. For example, the ITR sequences, which can form a hairpin structure, play roles in excision from the plasmid after transfection, replication of the vector genome and integration and rescue from a host cell genome.

The AAV expression cassettes of the disclosure may comprise a 5′ ITR and a 3′ ITR. The ITR sequences may be about 110 to about 160 nucleotides in length, for example 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 nucleotides in length. In some embodiments, the ITR sequences may be about 141 nucleotides in length. In some embodiments, the 5′ ITR is the same length as the 3′ ITR. In some embodiments, the 5′ ITR and the 3′ ITR have different lengths. In some embodiments, the 5′ ITR is longer than the 3′ ITR, and in other embodiments, the 3′ ITR is longer than the 5′ ITR.

The ITRs may be isolated or derived from the genome of any AAV, for example the AAVs listed in Table 1. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AA Vrh74, Avian AAV or Bovine AAV. In some embodiments, at least one of the 5′ ITR and the 3′ITR may be a wildtype or mutated ITR isolated derived from a member of another parvovirus species besides AAV. For example, in some embodiments, an ITR may be a wildtype or mutant ITR isolated or derived from bocavirus or parvovirus B19.

In some embodiments, the ITR comprises a modification to promote production of a scAAV. In some embodiments, the modification to promote production of a scAAV is deletion of the terminal resolution sequence (TRS) from the ITR. In some embodiments, the 5′ ITR is a wildtype ITR, and the 3′ ITR is a mutated ITR lacking the terminal resolution sequence. In some embodiments, the 3′ ITR is a wildtype ITR, and the 5′ ITR is a mutated ITR lacking the terminal resolution sequence. In some embodiments, the terminal resolution sequence is absent from both the 5′ ITR and the 3′ITR. In other embodiments, the modification to promote production of a scAAV is replacement of an ITR with a different hairpin-forming sequence, such as a shRNA-forming sequence.

In some embodiments, the 5′ ITR may comprise the sequence of SEQ ID NO: 1, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 3′ ITR may comprise the sequence of SEQ ID NO: 2, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 5′ ITR comprises the sequence of SEQ ID NO: 1, and the 3′ ITR comprises the sequence of SEQ ID NO: 2.

In some embodiments, the AAV expression cassettes comprise one or more “surrogate” ITRs, i.e., non-ITR sequences that serve the same function as ITRs. See, e.g., Xie, J. et al., Mol. Ther., 25 (6): 1363-1374 (2017). In some embodiments, an ITR in an AAV expression cassette is replaced by a surrogate ITR. In some embodiments, the surrogate ITR comprises a hairpin-forming sequence. In some embodiments, the surrogate ITR is a short hairpin (sh) RNA-forming sequence.

### (ii) Activity-Dependent Promoters

In some embodiments, the AAV expression cassettes described herein comprise a promoter. In some embodiments, the promoter is a tissue-specific promoter. In some embodiments, the promoter is a synthetic promoter. In some embodiments, the promoter may comprise a nucleic acid sequence derived from an endogenous promoter and/or an endogenous enhancer. In some embodiments, the promoter may comprise a nucleic acid sequence derived from the region upstream of the initiation codon of a gene. The nucleic acid sequence may lie within 1 base pair (bp) to within about 8000 bps upstream of the initiation codon, for example, within about 10 bps, 50 bps, 100 bps, 500 bps, 1000 bps, 1500 bps, 2000 bps, 2500 bps, 3000bps, 3500 bps, 4000 bps, 4500 bps, 5000 bps, 5500 bps, 6000 bps, 6500 bps, 7000 bps, 7500bps, or 8000 bps, inclusive of all subranges and values that lie therebetween, upstream of the initiation codon.

Without being bound by any theory, it is thought that MECP2 function contributes to expression of target genes involved in neuronal function. The expression of these target genes is affected when MECP2 is mutated. Restoring the expression of these target genes in a MECP2 independent manner may alleviate the symptoms of diseases associated with, or caused by, MECP2 mutations. Accordingly, in some embodiments, the promoter is a MECP2-independent promoter. As used herein, a “MECP2-independent promoter” refers to a promoter whose function is not impaired by reduced MECP2 function, or loss of MECP2 function.

Without being bound by any theory, it is thought that constitutive expression of target genes involved in neuronal function may be detrimental. Accordingly, in some embodiments, the promoter is an inducible promoter. In some embodiments, the function of the promoter is dependent on the presence of one or more stimuli. In some embodiments, the promoter is an activity-dependent promoter. As used herein, the function of an “activity-dependent promoter”, interchangeably used with “neuronal activity-dependent promoter”, is induced by neuronal activity. In some embodiments, the activity-dependent promoter is induced by the influx of calcium ions into the neuron. In some embodiments, the activity-dependent promoter is used to drive the expression of any target gene that when expressed constitutively in a cell (such as, a neuron), is detrimental.

In some embodiments, the promoter comprises a nucleic acid sequence derived from a promoter and/or an enhancer of a neuronal immediate early gene. The identity of the neuronal immediate early gene is not limited, and may refer to any neuronal immediate early gene known in the art, or identified in the future. Non-limiting examples of neuronal immediate early genes include Arc gene, the c-fos gene, and the egr-1 gene. Therefore, in some embodiments, the promoter comprises a nucleic acid sequence derived from the promoter and/or an enhancer of Arc gene, the c-fos gene, or the egr-I gene. In some embodiments, the promoter comprises a nucleic acid sequence derived from the promoter and/or an enhancer of a neurotrophin gene. Non-limiting examples of neurotrophin genes include bdnf (brain-derived neurotrophic factor) gene, ngf (nerve growth factor) gene, neutrophin-3 gene and neurotrophin-4 gene.

In some embodiments, the promoter is capable of binding to one or more neuronal activity-dependent transcription factors. As used herein, a “neuronal activity dependent transcription factor” is a transcription factor that is activated in response to neuronal activity. In some embodiments the neuronal activity-dependent transcription factor promotes gene expression in response to neuronal activity. In some embodiments, the neuronal activity-dependent transcription factor represses gene expression in response to neuronal activity. In some embodiments, the neuronal activity-dependent transcription factor is activated by calcium-dependent kinase cascades.

In some embodiments, the promoter comprises one or more nucleic acid sequences that are capable of binding to one or more neuronal activity-dependent transcription factors. Non-limiting examples of neuronal activity-dependent transcription factors include cAMP-responsive element binding protein (CREB), myocyte enhancer factor 2 (MEF2), serum response factor (SRF), or Elk-1. In some embodiments, the promoter is capable of binding to more than one neuronal activity-dependent transcription factor, for example, the promoter is capable of binding to 2, 3, 4, or 5 neuronal activity-dependent transcription factors. In some embodiments, the promoter is capable of binding to two neuronal activity-dependent transcription factors. In some embodiments, the promoter is capable of binding to three neuronal activity-dependent transcription factors. In some embodiments, the promoter is capable of binding to the following neuronal activity-dependent transcription factors: CREB, MEF2 and SRF.

In some embodiments, the promoter comprises one or more response elements. As used herein, a “response element” is a region of the promoter that contributes to driving gene expression in the presence of a stimulant. In some embodiments, the response element is critical for the promoter to drive gene expression in the presence of the stimulant. Non-limiting examples of stimulant include hormones; environmental cues, such as heat or light; and chemical ions such as calcium. In some embodiments, the promoter comprises a response element that drives gene expression in the presence of calcium. In some embodiments, the promoter comprises a response element that is present in the promoters of neuronal immediate early genes. In some embodiments, the promoters comprises a response element that is present in the promoter of the Arc gene, the c-fos gene, and/or the egr-1 gene.

In some embodiments, the response element comprises one or more of the following: cyclic AMP response element (CRE), a serum response element (SRE), a synaptic activity response element (SARE), and a MEF2 response element (MRE). In some embodiments, the promoter comprises one or more CREs; one or more SREs; one or more SAREs; one or more MREs, or any combination thereof. In some embodiments, the response element binds to one or more neuronal activity dependent transcription factors. In some embodiments, the CRE binds to CREB. In some embodiments, the SRE binds to SRF. In some embodiments, the MRE binds to MEF2. In some embodiments, the SARE binds to CREB, MEF2 and SRF.

In some embodiments, the promoter comprises 1-20 SAREs, for example, 1, 2, 3, 4, 5, 6, 7, 8, 9, 10, 11, 12, 13, 14, 15, 16, 17, 18, 19, or 20 SAREs. In some embodiments, the promoter comprises 1 SARE. In some embodiments, the promoter comprises 5 SAREs. In some embodiments, the SARE is a mouse SARE. In some embodiments, the SARE is a human synaptic activity response element (hSARE). In some embodiments, the hSARE comprises a nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 11. In some embodiments, the hSARE comprises a nucleic acid sequence of SEQ ID NO: 11, or a sequence at least 90% identical thereto.

In some embodiments, the promoter comprises a nucleic acid sequence derived from the promoter of the Arc gene. In some embodiments, the Arc gene is the mouse Arc gene. In some embodiments, the Arc gene is the human Arc gene. In some embodiments, the promoter comprises a region spanning nucleic acids −300 to +300 of the Arc gene, or any subregion thereof. In some embodiments, the promoter comprises a minimal Arc gene promoter (ArcMin). In some embodiments, the human ArcMin (hArcMin) comprises a short upstream sequence and 5′ UTR (−276 to +208) of the Arc gene. In some embodiments, the mouse ArcMin (mArcMin) comprises a short upstream sequence and 5′ UTR (−222 to +198) of the Arc gene. In some embodiments, the hArcMin comprises a nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 12. In some embodiments, the hArcMin comprises a nucleic acid sequence of SEQ ID NO: 12, or a sequence at least 90% identical thereto.

In some embodiments, the promoter comprises hArcMin and at least one hSARE. The number of hSAREs in the promoter is not limited, and may be in the range of 1 hSARE to 20 hSAREs, such as, 1, 2, 3, 4, 5, 6, 7, 8, 9, 10, 11, 12, 13, 14, 15, 16, 17, 18, 19, or 20 hSARES. In some embodiments, the promoter comprises hArcMin and one hSARE (called SARE-ArcMin). In some embodiments, the SARE-ArcMin comprises a nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 6. In some embodiments, the SARE-ArcMin comprises a nucleic acid sequence of SEQ ID NO: 6, or a sequence at least 90% identical thereto. In some embodiments, the promoter comprises hArcMin and 5 hSAREs (called E-SARE). In some embodiments, the E-SARE comprises a nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie there between) to the nucleic acid sequence of SEQ ID NO: 16. In some embodiments, the E-SARE comprises a nucleic acid sequence of SEQ ID NO: 16, or a sequence at least 90% identical thereto. Further details regarding hArcMin and hSARE are provided in Kawashima et al., Nat Methods 10, 889-895 (2013), Kawashima et al., Front Neural Circuits 2014 Apr. 23; 8:37, and Kawashima et al., PNAS Jan. 6, 2009 106(1)316-32, each of which is incorporated herein by reference in its entirety.

In some embodiments, the promoter induces a level of gene expression that higher than an endogenous promoter of a neuronal immediate early gene, such as Arc gene, the c-fos gene, or the egr-1 gene. In some embodiments, the promoter induces a level of gene expression that is at least 1.5 fold higher than an endogenous promoter of a neuronal immediate early gene, such as Arc gene, the c-fos gene, or the egr-l gene. In some embodiments, the promoter induces a level of gene expression that is about 1.5-fold to about 100-fold (for example, about 2-fold, about 3-fold, about 4-fold, about 5-fold, about 10-fold, about 15-fold, about 20-fold, about 30-fold, about 40-fold, about 50-fold, about 60-fold, about 70-fold, about 80-fold, about 90-fold, or about 100-fold) higher than an endogenous promoter of a neuronal immediate early gene, such as Arc gene, the c-fos gene, or the egr-1 gene.

In some embodiments, the promoter comprises a nucleic acid sequence derived from one or more promoters commonly used in the art for gene expression. For instance, in some embodiments, the promoter further comprises a nucleic acid sequence derived from the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the dihydrofolate reductase (DHFR) promoter, and the phosphoglycerol kinase (PGK) promoter. In some embodiments, the promoter comprises a nucleic acid sequence derived from the chicken β-actin (CBA) promoter, the EF-1 alpha promoter, or the EF-1 alpha short promoter. In some embodiments, the promoter comprises a sequence selected from any one of SEQ ID NOs: 17-20, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between).

In some embodiments, the AAV expression cassettes described herein further comprise an enhancer. The enhancer may be, for example, the CMV enhancer. In some embodiments, the enhancer comprises the sequence of SEQ ID NO: 21, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween).

In some embodiments, the promoter further comprises a nucleic acid sequence derived from any one or more of the following promoters: HMG-COA reductase promoter; sterol regulatory element 1 (SRE-1); phosphoenol pyruvate carboxy kinase (PEPCK) promoter; human C-reactive protein (CRP) promoter; human glucokinase promoter; cholesterol 7-alpha hydroylase (CYP-7) promoter; beta-galactosidase alpha-2,6 sialyltransferase promoter; insulin-like growth factor binding protein (IGFBP-1) promoter; aldolase B promoter; human transferrin promoter; collagen type I promoter; prostatic acid phosphatase (PAP) promoter; prostatic secretory protein of 94 (PSP 94) promoter; prostate specific antigen complex promoter; human glandular kallikrein gene promoter (hgt-1); the myocyte-specific enhancer binding factor MEF-2; muscle creatine kinase promoter; pancreatitis associated protein promoter (PAP); elastase 1 transcriptional enhancer; pancreas specific amylase and elastase enhancer promoter; pancreatic cholesterol esterase gene promoter; uteroglobin promoter; cholesterol side-chain cleavage (SCC) promoter; gamma-gamma enolase (neuron-specific enolase, NSE) promoter; neurofilament heavy chain (NF-H) promoter; human CGL-1/granzyme B promoter; the terminal deoxy transferase (TdT), lambda 5, VpreB, and lck (lymphocyte specific tyrosine protein kinase p561ck) promoter; the humans CD2 promoter and its 3′ transcriptional enhancer; the human NK and T cell specific activation (NKG5) promoter; pp60c-src tyrosine kinase promoter; organ-specific neoantigens (OSNs), mw 40 kDa (p40) promoter; colon specific antigen-P promoter; human alpha-lactalbumin promoter; phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter.

### (iii) Rett Syndrome-Associated Gene

As used herein, a “Rett Syndrome-associated gene” refers to any gene in a subject with Rett syndrome which can be targeted by gene therapy to alleviate at least one sign or symptom of Rett Syndrome. In some embodiments, the level of the protein encoded by the Rett Syndrome-associated gene is reduced or undetectable in subjects with Rett syndrome. In some embodiments, the levels of the protein encoded by the Rett Syndrome-associated gene decreases upon the onset of at least one symptom of Rett Syndrome. In some embodiments, the levels of the protein encoded by the Rett Syndrome-associated gene increases in normal post-natal development and/or neuronal development. In some embodiments, the Rett Syndrome-associated gene encodes a protein that contributes to normal neuronal function. In some embodiments, the Rett Syndrome-associated gene encodes a protein that is involved in promoting and/or maintaining synaptic plasticity. In some embodiments, the Rett Syndrome-associated gene is a neurotrophin. In some embodiments, mutations in the Rett Syndrome-associated gene, or loss of function of the Rett Syndrome-associated gene, is present in subjects with Rett Syndrome. In some embodiments, mutations in the Rett Syndrome-associated gene, or loss of function of the Rett Syndrome-associated gene, causes Rett Syndrome. In some embodiments, mutations in the Rett Syndrome-associated gene, or loss of function of the Rett Syndrome-associated gene, particularly in forebrain excitary neurons, causes symptoms similar to the loss of mecp2 gene. The type of mutation is not limited, and may be an insertion, deletion, duplication and/or substitution.

The disclosure provides AAV expression cassettes comprising a Rett Syndrome-associated gene. In some embodiments, an AAV expression cassette comprises a Rett Syndrome-associated gene which encodes a protein, including therapeutic (e.g., for medical or veterinary uses) or immunogenic (e.g., for vaccines) polypeptide. In some embodiments, AAV expression cassette comprises a mammalian Rett Syndrome-associated gene. In some embodiments, the AAV expression cassette comprises a human Rett Syndrome-associated gene. In some embodiments, the AAV expression cassette comprises a Rett Syndrome-associated gene that encodes brain-derived neurotrophic factor (BDNF), insulin-like growth factor 1 (IGF1), methyl-CpG-binding protein 2 (MECP2), Huntingtin protein, Huntington-associated protein 1, Orthodenticle homeobox 2 (OTX-2), FXYD Domain Containing Ion Transport Regulator 1 (FXYD1), Neurexin-2-alpha (NRXN2), or Protein Kinase C Gamma (PRKCG). In some embodiments, the Rett Syndrome-associated gene is a gene selected from the group consisting of KCNA1 (Potassium Voltage-Gated Channel Subfamily A Member 1), GABRA1 (Gamma-Aminobutyric Acid Type A Receptor Subunit Alpha1), MAPK1 (Mitogen-Activated Protein Kinase 1), NRXN2 (Neurexin 2), RBFOX1 (RNA Binding Fox-1 Homolog 1), GNAO1 (G Protein Subunit Alpha 01), NCAN (Neurocan), PRKCG (Protein Kinase C Gamma), KCNJ4 (Potassium Inwardly Rectifying Channel Subfamily J Member 4), CAMK2B (Calcium/Calmodulin Dependent Protein Kinase II Beta), EFNB3 (Ephrin B3), GABBR1 (Gamma-Aminobutyric Acid Type B Receptor Subunit 1), LY6H (Lymphocyte Antigen 6 Family Member H), KCNA2 (Potassium Voltage-Gated Channel Subfamily A Member 2), and NEFL (Neurofilament Light Chain).

In some embodiments, the Rett-syndrome-associated gene encodes brain-derived neurotrophic factor (BDNF). BDNF is a neurotrophin that functions in supporting survival and growth of neurons, synaptic development, and plasticity. BDNF expression is altered in Rett syndrome. Loss of function of bdnf results in phenotypes similar to loss of function of mecp2, and BDNF function is affected upon loss of mecp2 function. Further details about BDNF function are provided in Eduardo E. Benarroch, Neurology April 2015, 84 (16) 1693-1704;Chang et al. Neuron. 2006; 49(3):341-348; and Zhou et al., Neuron 52, 255-269, Oct. 19, 2006, each of which is incorporated herein by reference in its entirety. In some embodiments, the BDNF is a human BDNF. In some embodiments, the BDNF is encoded by a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie there between) to the nucleic acid sequence of SEQ ID NO: 7. In some embodiments, the BDNF is encoded by a nucleic acid sequence of SEQ ID NO: 7, or a sequence at least 90% identical thereto.

There are two alternate polyadenylated transcription stop sites in BDNF, which generate two distinct populations of bdnf mRNA, with either short 3′ UTR (about 0.35 kb long), or long 3′ UTR (about 2.85 kb long). The bdnf mRNA variant with the short 3′ UTR is mostly localized to the cell body of neurons, whereas the bdnf mRNA variant with the long 3′ UTR is also localized to dendrites. In some embodiments, the AAV expression cassette comprises BDNF short 3′ UTR, or the BDNF long 3′ UTR. In some embodiments, the BDNF short 3′ UTR, or the BDNF long 3′ UTR is present between the stop codon and the 3′ ITR.

In some embodiments, the BDNF short 3′ UTR comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 8. In some embodiments, the BDNF short 3′ UTR comprises a nucleic acid sequence of SEQ ID NO: 8, or a sequence at least 90% identical thereto. In some embodiments, the BDNF short 3′ UTR comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 10. In some embodiments, the BDNF long 3′ UTR comprises a nucleic acid sequence of SEQ ID NO: 10, or a sequence at least 90% identical thereto.

In some embodiments, the AAV expression cassette comprises a Kozak sequence. The Kozak sequence is a nucleic acid sequence that functions as a protein translation initiation site in many eukaryotic mRNA transcripts. In some embodiments, the Kozak sequence overlaps with the start codon. In some embodiments, the Kozak sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 14 or SEQ ID NO: 15 (acagccacc). In some embodiments, the Kozak sequence comprises a nucleic acid sequence of SEQ ID NO: 14, or a sequence at least 90% identical thereto; or a nucleic acid sequence of SEQ ID NO: 15 (acagccacc), or a sequence at least 90% identical thereto.

### (iv) Polyadenylation (PolyA) Signal

Polyadenylation signals are nucleotide sequences found in nearly all mammalian genes and control the addition of a string of approximately 200 adenosine residues (the poly(A) tail) to the 3′ end of the gene transcript. The poly(A) tail contributes to mRNA stability, and mRNAs lacking the poly(A) tail are rapidly degraded. There is also evidence that the presence of the poly(A) tail positively contributes to the translatability of mRNA by affecting the initiation of translation.

In some embodiments, the AAV expression cassettes of the disclosure comprise a polyadenylation signal. The polyadenylation signal may be selected from the polyadenylation signal of simian virus 40 (SV40), rBG. α-globin, β-globin, human collagen, human growth hormone (hGH), polyoma virus, human growth hormone (hGH) and bovine growth hormone (bGH).

In some embodiments, the AAV expression cassette comprises a bGH polyadenylation signal. In some embodiments, the bGH polyadenylation signal comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 9. In some embodiments, the bGH polyadenylation signal comprises a nucleic acid sequence of SEQ ID NO: 9, or a sequence at least 90% identical thereto.

In some embodiments, the polyadenylation signal is the SV40 polyadenylation signal. In some embodiments, the polyadenylation signal is the rBG polyadenylation signal. In some embodiments, the polyadenylation signal comprises the sequence of SEQ ID NO: 22 or SEQ ID NO: 23. In some embodiments, the polyadenylation signal comprises a sequence at least 90%, at least 95%, at least 96%, at least 97%, at least 98%, or at least 99% identical to the sequence of SEQ ID NO: 22 or SEQ ID NO: 23.

### (v) Stuffer Sequences

AAV vectors typically accept inserts of DNA having a defined size range which is generally about 4 kb to about 5.2 kb, or slightly more. Thus, for shorter sequences, it may be necessary to include additional nucleic acid in the insert fragment in order to achieve the required length which is acceptable for the AAV vector. Accordingly, in some embodiments, the AAV expression cassettes of the disclosure may comprise a stuffer sequence. The stuffer sequence may be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500,or 4,500-5,000, or more nucleotides in length. The stuffer sequence can be located in the cassette at any desired position such that it does not prevent a function or activity of the vector. In some embodiments, the stuffer sequence is located upstream of the 3′ UTR. For instance, in some embodiments, the stuffer sequence is present between the BDNF short 3′ UTR and the 3′ ITR, or between the bGH poly A signal and the 3′ ITR.

In some embodiments, the AAV cassette comprises at least one stuffer sequence. In some embodiments, the stuffer sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 13. In some embodiments, the stuffer sequence comprises a nucleic acid sequence of SEQ ID NO: 13, or a sequence at least 90% identical thereto.

### (vi) Intronic Sequences

In some embodiments, the AAV expression cassettes of the disclosure may comprise an intronic sequence. Inclusion of an intronic sequence may enhance expression compared with expression in the absence of the intronic sequence.

In some embodiments, the intronic sequence is a hybrid or chimeric sequence. In some embodiments, the intronic sequence is isolated or derived from an intronic sequence of one or more of SV40, β-globin, chicken beta-actin, minute virus of mice (MVM), factor IX, and/or human IgG (heavy or light chain). In some embodiments, the intronic sequence is chimeric. In some embodiments, the intronic sequence comprises the sequence of SEQ ID NO: 24 or SEQ ID NO: 25. In some embodiments, the intronic sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 24 or SEQ ID NO: 25.

### (vii) Exemplary AAV Expression Cassettes

In some embodiments, the AAV expression cassette comprises a 5′ inverted terminal repeat (ITR), a transgene, and a 3′ ITR. In some embodiments, the AAV expression cassette comprises SEQ ID NO: 1 (5′ ITR), SEQ ID NO: 7 (BDNF) and SEQ ID NO: 2 (3′ ITR).

In some embodiments, the AAV expression cassette comprises a 5′ ITR, a promoter, a transgene, and a 3′ ITR. In some embodiments, the AAV expression cassette comprises SEQ ID NO: 1 (5′ ITR), SEQ ID NO: 6 (hSARE-ArcMin), SEQ ID NO: 7 (BDNF) and SEQ ID NO: 2 (3′ ITR). In some embodiments, the AAV expression cassette comprises SEQ ID NO: 1 (5′ ITR), SEQ ID NO: 16 (E-SARE), SEQ ID NO: 7 (BDNF) and SEQ ID NO: 2 (3′ ITR).

In some embodiments, the AAV expression cassette comprises a 5′ ITR, a promoter, a transgene, a polyadenylation sequence and a 3′ ITR. In some embodiments, the AAV expression cassette comprises SEQ ID NO: 1 (5′ ITR), SEQ ID NO: 6 (hSARE-ArcMin), SEQ ID NO: 7 (BDNF), SEQ ID NO: 9 (bGH polyadenylation sequence) and SEQ ID NO: 2 (3′ ITR). In some embodiments, the AAV expression cassette comprises SEQ ID NO: 1 (5′ ITR), SEQ ID NO: 16 (E-SARE), SEQ ID NO: 7 (BDNF), SEQ ID NO: 9 (bGH polyadenylation sequence) and SEQ ID NO: 2 (3′ ITR).

In some embodiments, the AAV expression cassette comprises a 5′ ITR, a promoter, Kozak sequence, a transgene, and a 3′ ITR. In some embodiments, the AAV expression cassette comprises SEQ ID NO: 1 (5′ ITR), SEQ ID NO: 6 (hSARE-ArcMin), SEQ ID NO: 14 or 15 (acagccacc) (Kozak sequence), SEQ ID NO: 7 (BDNF), and SEQ ID NO: 2 (3′ ITR). In some embodiments, the AAV expression cassette comprises SEQ ID NO: 1 (5′ ITR), SEQ ID NO: 16 (E-SARE), SEQ ID NO: 14 or 15 (acagccacc) (Kozak sequence), SEQ ID NO: 7 (BDNF), and SEQ ID NO: 2 (3′ ITR).

In some embodiments, the AAV expression cassette comprises a 5′ ITR, a promoter, Kozak sequence, a transgene, a polyadenylation sequence and a 3′ ITR. In some embodiments, the AAV expression cassette comprises SEQ ID NO: 1 (5′ ITR), SEQ ID NO: 6 (hSARE-ArcMin), SEQ ID NO: 14 or 15 (acagccacc) (Kozak sequence), SEQ ID NO: 7 (BDNF), SEQ ID NO: 9 (bGH polyadenylation sequence) and SEQ ID NO: 2 (3′ ITR). In some embodiments, the AAV expression cassette comprises SEQ ID NO: 1 (5′ ITR), SEQ ID NO: 16 (E-SARE), SEQ ID NO: 14 or 15 (acagccacc) (Kozak sequence), SEQ ID NO: 7 (BDNF), SEQ ID NO: 9 (bGH polyadenylation sequence) and SEQ ID NO: 2 (3′ ITR).

In some embodiments, the AAV expression cassette comprises a 5′ ITR, a promoter, a transgene, a 3′ UTR and a 3′ ITR. In some embodiments, the AAV expression cassette comprises SEQ ID NO: 1 (5′ ITR), SEQ ID NO: 6 (hSARE-ArcMin), SEQ ID NO: 7 (BDNF), SEQ ID NO: 8 (BDNF short 3′UTR), and SEQ ID NO: 2 (3′ ITR). In some embodiments, the AAV expression cassette comprises SEQ ID NO: 1 (5′ ITR), SEQ ID NO: 16 (E-SARE), SEQ ID NO: 7 (BDNF), SEQ ID NO: 8 (BDNF short 3′UTR), and SEQ ID NO: 2 (3′ ITR). In some embodiments, the AAV expression cassette comprises SEQ ID NO: 1 (5′ ITR), SEQ ID NO: 6 (hSARE-ArcMin), SEQ ID NO: 7 (BDNF), SEQ ID NO: 10 (BDNF long 3′UTR), and SEQ ID NO: 2 (3′ ITR). In some embodiments, the AAV expression cassette comprises SEQ ID NO: 1 (5′ ITR), SEQ ID NO: 16 (E-SARE), SEQ ID NO: 7 (BDNF), SEQ ID NO: 10 (BDNF long 3′UTR), and SEQ ID NO: 2 (3′ ITR).

In some embodiments, the AAV expression cassette comprises a 5′ ITR, a promoter, a transgene, a 3′ UTR, a polyadenylation sequence and a 3′ ITR. In some embodiments, the AAV expression cassette comprises SEQ ID NO: 1 (5′ ITR), SEQ ID NO: 6 (hSARE-ArcMin), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR). In some embodiments, the AAV expression cassette comprises SEQ ID NO: 1 (5′ ITR), SEQ ID NO: 16 (E-SARE), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR).

In some embodiments, the AAV expression cassette comprises a 5′ ITR, a promoter, Kozak sequence, a transgene, a 3′ UTR and a 3′ ITR. In some embodiments, the AAV expression cassette comprises SEQ ID NO: 1 (5′ ITR), SEQ ID NO: 6 (hSARE-ArcMin), SEQ ID NO: 14 or 15 (acagccacc) (Kozak sequence), SEQ ID NO: 7 (BDNF), SEQ ID NO: 8 or 10 (BDNF short or long 3′UTR), and SEQ ID NO: 2 (3′ ITR). In some embodiments, the AAV expression cassette comprises SEQ ID NO: 1 (5′ ITR), SEQ ID NO: 16 (E-SARE), SEQ ID NO: 14 or 15 (acagccacc) (Kozak sequence), SEQ ID NO: 7 (BDNF), SEQ ID NO: 8 or 10 (BDNF short or long 3′UTR), and SEQ ID NO: 2 (3′ ITR).

In some embodiments, the AAV expression cassette comprises a 5′ ITR, a promoter, a Kozak sequence, a transgene, a 3′ UTR, a polyadenylation sequence and a 3′ ITR. In some embodiments, the AAV expression cassette comprises SEQ ID NO: 1 (5′ ITR), SEQ ID NO: 6 (hSARE-ArcMin), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR). In some embodiments, the AAV expression cassette comprises SEQ ID NO: 1 (5′ ITR), SEQ ID NO: 16 (E-SARE), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR).

In some embodiments, the AAV expression cassette comprises a 5′ ITR, a stuffer sequence, a promoter, a Kozak sequence, a transgene, a 3′ untranslated region (3′ UTR), a polyadenylation sequence, and a 3′ ITR. In some embodiments, the AAV expression cassette comprises SEQ ID NO: 1 (5′ ITR), SEQ ID NO: 13 (stuffer sequence), SEQ ID NO: 6 (hSARE-ArcMin), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR). In some embodiments, the AAV expression cassette comprises SEQ ID NO: 1 (5′ ITR), SEQ ID NO: 13 (stuffer sequence), SEQ ID NO: 16 (E-SARE), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence) and SEQ ID NO: 2 (3′ ITR).

In some embodiments, the AAV expression cassette comprises a 5′ ITR, a promoter, a Kozak sequence, a transgene, a 3′ untranslated region (3′ UTR), a polyadenylation sequence, a stuffer sequence and a 3′ ITR. In some embodiments, the AAV expression cassette comprises SEQ ID NO: 1 (5′ ITR), SEQ ID NO: 6 (hSARE-ArcMin), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence), SEQ ID NO: 13 (stuffer sequence), and SEQ ID NO: 2 (3′ ITR). In some embodiments, the AAV expression cassette comprises SEQ ID NO: 1 (5′ ITR), SEQ ID NO: 13 (stuffer sequence), SEQ ID NO: 16 (E-SARE), SEQ ID NO: 14 or 15 (acagccacc) (Kozak sequence), SEQ ID NO: 7 (BDNF), SEQ ID NO: 8 or 10 (BDNF short or long 3′UTR), SEQ ID NO: 9 (bGH polyadenylation sequence), SEQ ID NO: 13 (stuffer sequence), and SEQ ID NO: 2 (3′ ITR).

### AAV Production Methods

The AAV expression cassettes described herein may be incorporated into a vector (e.g., a plasmid or a bacmid) using standard molecular biology techniques. The disclosure provides vectors comprising any one of the AAV expression cassettes described herein. The vector (e.g., plasmid or bacmid) may further comprise one or more genetic elements used during production of AAV, including, for example, AAV rep and cap genes, and helper virus protein sequences.

The AAV expression cassettes, and vectors (e.g., plasmids) comprising the AAV expression cassettes described herein may be used to produce recombinant AAV vectors.

The disclosure provides methods for producing a recombinant AAV vector comprising contacting an AAV producer cell (e.g., an HEK293 cell) with an AAV expression cassette, or vector (e.g., plasmid) of the disclosure. The disclosure further provides cells comprising any one of the AAV expression cassettes, or vectors disclosed herein. In some embodiments, the method further comprises contacting the AAV producer cell with one or more additional plasmids encoding, for example, AAV rep and cap genes, and helper virus protein sequences. In some embodiments, a method for producing a recombinant AAV vector comprises contacting an AAV producer cell (e.g., an insect cell such as a Sf9 cell) with at least one insect cell-compatible vector comprising an AAV expression cassette of the disclosure. An “insect cell-compatible vector” is any compound or formulation (biological or chemical), which facilitates transformation or transfection of an insect cell with a nucleic acid. In some embodiments, the insect cell-compatible vector is a baculoviral vector. In some embodiments, the method further comprises maintaining the insect cell under conditions such that AAV is produced.

The disclosure provides recombinant AAV vectors produced using any one of the methods disclosed herein. The recombinant AAV vectors produced may be of any serotype, for example AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the recombinant AAV vectors produced may comprise one or more amino acid modifications (e.g., substitutions and/or deletions) compared to the native AAV capsid. For example, the recombinant AAV vectors may be modified AAV vectors derived from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vector is a single-stranded AAV (ssAAV). In some embodiments, the recombinant AAV vector is a self-complementary AAV (scAAV).

In some embodiments, the AAV vector comprises a capsid protein of AAV1, AAV2,AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the AAV vector comprises a capsid protein with one or more substitutions or mutations, as compared to a wild type AAV capsid protein. The recombinant AAV vectors disclosed herein may be used to transduce target cells with the transgene sequence, for example by contacting the recombinant AAV vector with a target cell.

### Methods of Expression and Treatment

The disclosure provides compositions comprising any one of the nucleic acids, AAV expression cassettes, plasmids, cells, or recombinant AAV vectors disclosed herein. In some embodiments, the compositions disclosed herein comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

The disclosure provides methods of delivering a Rett Syndrome-associated gene into a cell by contacting the cell with any one of the any one of the nucleic acids, AAV expression cassettes, plasmids, recombinant AAV vectors, or compositions disclosed herein. In some embodiments, the cell is a dividing cell, such as a cultured cell in cell culture. In some embodiments, the cell is a non-dividing cell. In some embodiments, the Rett Syndrome-associated gene is delivered to the cell in vitro, e.g., to produce the Rett Syndrome-associated polypeptide in vitro or for ex vivo gene therapy.

In some embodiments, the Rett Syndrome-associated gene is delivered to a subject in need thereof, e.g., to express an immunogenic or therapeutic polypeptide. In this manner, the polypeptide or functional RNA can be produced in vivo in the subject. Thus, the disclosure provides methods of expressing a Rett-syndrome-associated gene in a subject in need thereof, comprising administering to the subject a therapeutically effective amount of any one of the nucleic acids, AAV expression cassettes, plasmids, cells, recombinant AAV vectors, or compositions disclosed herein. The disclosure also provides methods for treating and/or delaying the onset of at least one symptom of Rett Syndrome in a subject. In some embodiments, the methods comprise administering to the subject a therapeutically effective amount of any one of the nucleic acid of any one of the nucleic acids, AAV expression cassettes, plasmids, cells, recombinant AAV vectors, or compositions disclosed herein. In some embodiments, the subject has Rett Syndrome. In some embodiments, the subject has a high risk of developing Rett Syndrome; for example, the subject is a newborn who is identified as carrying a mutation in the mecp2 gene. In some embodiments, the Rett-syndrome-associated gene is targeted by gene therapy to increase its expression and/or function. In some embodiments, the Rett-syndrome-associated gene is targeted by gene therapy to decrease its expression and/or function.

The disclosure provides methods of treating and/or delaying the onset of a MECP2-associated disease in a subject, comprising administering to the subject a therapeutically effective amount of any one of the nucleic acids, AAV expression cassettes, plasmids, cells, recombinant AAV vectors, or compositions disclosed herein. As used herein, a “MECP2-associated disease” is a disease which is correlated with, or caused by, genetic changes (for example, one or more deletions, insertions, duplications and/or substitutions) to the mecp2 gene, as compared to the wild type mecp2 gene, and/or alterations to the expression and/or activity of the MECP2 protein, as compared with a wild type MECP2 protein.

In some embodiments, the MECP2-associated disease is MECP2 duplication syndrome, MECP2-related severe neonatal encephalopathy, PPM-X syndrome, or Rett syndrome. MECP2 duplication syndrome is caused by duplication of the MECP2 gene, and is characterized by intellectual disability, delayed development, and seizures. Duplication of the MECP2 gene leads to the production of extra MECP2 protein and an increase in protein function, leading to abnormal neuronal function. MECP2-related severe neonatal encephalopathy is caused by mutations in the mecp2 gene, most of which are single base pair insertions, deletions or substitutions. This condition almost exclusively affects males, and is characterized by small head size (microcephaly), movement disorders, breathing problems, and seizures. Mutations in the mecp2 gene can alter the structure of the MECP2 protein or reduce the amount of protein that is produced. PPM-X syndrome is a disease characterized by mild to severe intellectual disability, bipolar disorder, and a pattern of movement abnormalities. Approximately half of all cases of PPM-X syndrome are caused by one of eight mutations in mecp2 gene. These mutations either cause insertions, deletions or substitutions of amino acids in the MECP2 protein, or create a premature stop signal in mecp2 mRNA.

The disclosure further provides methods of treating and/or delaying the onset of a BDNF-associated disease in a subject, comprising administering to the subject a therapeutically effective amount of any one of the nucleic acids, AAV expression cassettes, plasmids, cells, recombinant AAV vectors, or compositions disclosed herein. As used herein, a “BDNF-associated disease” is a disease which is correlated with, or caused by, genetic changes to the bdnf gene, and/or changes to the expression and/or activity of the BDNF protein. In some embodiments, the BDNF-associated disease is a cognitive disorder, and/or a stress-related disorder. In some embodiments, the BDNF-associated disease is depression, obsessive-compulsive disorder, Alzheimer's disease, Huntington's disease, dementia, anorexia nervosa, bulimia nervosa, schizophrenia, epilepsy, post-traumatic stress disorder, bipolar disease, Rett Syndrome, major depressive disorder, or post-chemotherapy cognitive impairment.

In some embodiments, the Rett Syndrome-associated gene is expressed in the neurons of the subject. In some embodiments, the level of expression of the Rett Syndrome-associated gene is higher in neurons than in non-neuronal cells of the body. In some embodiments, the expression of the Rett Syndrome-associated gene is not detectable in non-neuronal cells. In some embodiments, the level of expression of the Rett Syndrome-associated gene is higher in neurons by at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) relative to that in non-neuronal cells of the body.

In some embodiments, the neurons are active neurons. In some embodiments, the level of expression of the Rett Syndrome-associated gene is higher in active neurons than in resting neurons of the body. In some embodiments, the expression of the Rett Syndrome-associated gene is not detectable in resting neurons. In some embodiments, the level of expression of the Rett Syndrome-associated gene is higher in active neurons by at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) relative to that in resting neurons.

In some embodiments, the Rett Syndrome-associated gene is expressed in the central nervous system (CNS) neurons of the subject. In some embodiments, the level of expression of the Rett Syndrome-associated gene is higher in CNS neurons by at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) relative to that in non-neuronal cells of the body.

In some embodiments, the level of expression of the Rett Syndrome-associated gene is higher in CNS neurons than in other non-CNS neurons, such as, peripheral nervous system (PNS) neurons of the body. In some embodiments, the expression of the Rett Syndrome-associated gene is not detectable in PNS neurons. In some embodiments, the level of expression of the Rett Syndrome-associated gene is higher in CNS neurons by at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) relative to that in other non-CNS neurons, such as, peripheral nervous system (PNS) neurons of the body.

Dosages of the recombinant AAV vector to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, about 10⁶, about 10⁷, about 10⁸, about 10⁹, about 10¹⁰, about 10¹¹, about 10¹², about 10¹³, about 10¹⁴, about 10¹⁵ transducing units, optionally about 10⁸ to about 10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, the administration is by injection into the central nervous system. The most suitable route in any given case will depend on the nature and severity of the condition being treated and/or prevented and on the nature of the particular vector that is being used.

Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid.

In some embodiments, the methods disclosed herein may comprise administering to the subject a therapeutically effective amount of any one of the nucleic acids, AAV expression cassettes, plasmids, cells, recombinant AAV vectors, or compositions disclosed herein in combination with one or more secondary therapies targeting Rett syndrome. In some embodiments, the methods of treating and/or delaying the onset of at least one symptom of Rett Syndrome in a subject disclosed herein may further comprise administering one or more secondary therapies targeting Rett syndrome. In some embodiments, the secondary therapy comprises administration of glatiramer acetate. Further details regarding the use of glatiramer acetate are provided in Djukic A et al., Pediatr Neurol. 2016 August; 61:51-7, the contents of which are incorporated herein by reference in its entirety.

In some embodiments, the secondary therapy comprises: administration of a drug to treat seizures, administration of a drug to treat reflux, or a combination thereof. In some embodiments, the secondary therapy comprises administration of a drug to treat seizures. Non-limiting examples of drugs to treat seizures include levetiracetam, valproic acid, oxcarbazepine, and lamotrigine. In some embodiments, the secondary therapy comprises administration of a drug to treat reflux. Non-limiting examples of drugs to treat reflux include H2 blockers, such as, cimetidine, ranitidine, nizatidine, and famotidine; proton pump inhibitors, such as, omeprazole, esomeprazole, lansoprazole, rabeprazole, pantoprazole, and dexlansoprazole; and motility agents, such as, low-dose erythromycin, benzamide, domperidone, and linaclotide.

The term administered “in combination,” as used herein, is understood to mean that two (or more) different treatments are delivered to the subject during the course of the subject's affliction with the disorder (such as, Rett syndrome), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” delivery.

In some embodiments, the treatment is more effective because of combined administration. For example, the second treatment is more effective; for e.g., an equivalent effect is seen with less of the second treatment, or the second treatment reduces symptoms to a greater extent, than would be seen if the second treatment were administered in the absence of the first treatment, or the analogous situation is seen with the first treatment. The effect of the two treatments can be partially additive, wholly additive, or greater than additive (synergistic).

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

The following examples, which are included herein for illustration purposes only, are not intended to be limiting.

### Example 1: Preparation of AAV Expression Cassettes

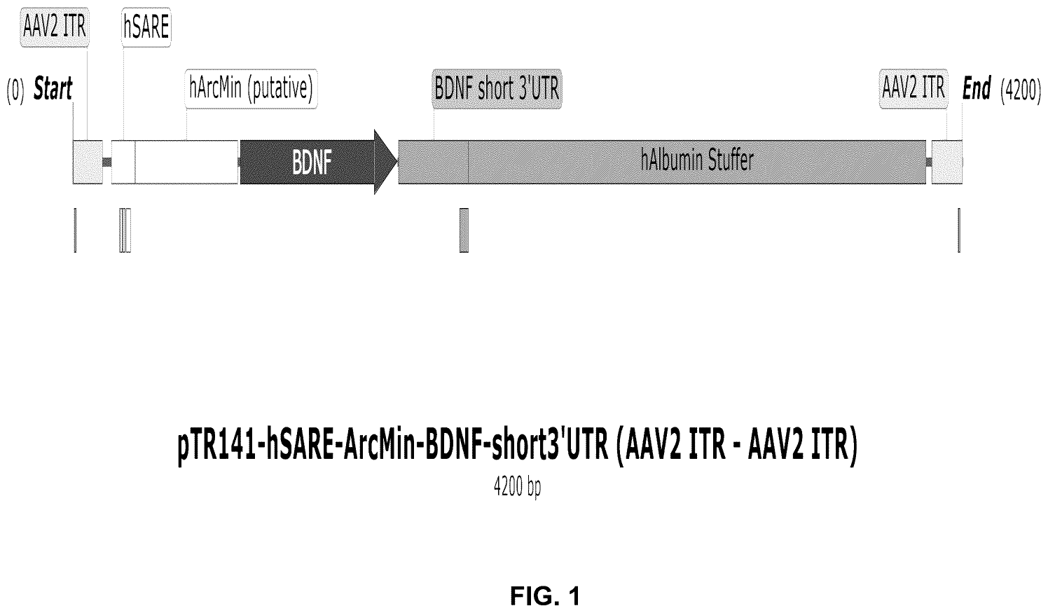

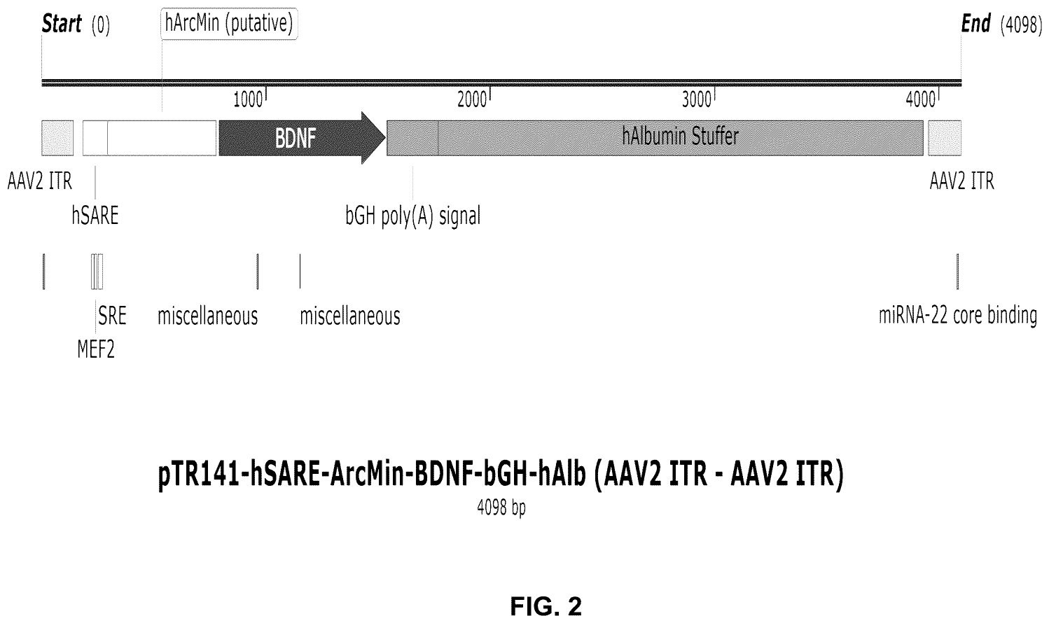

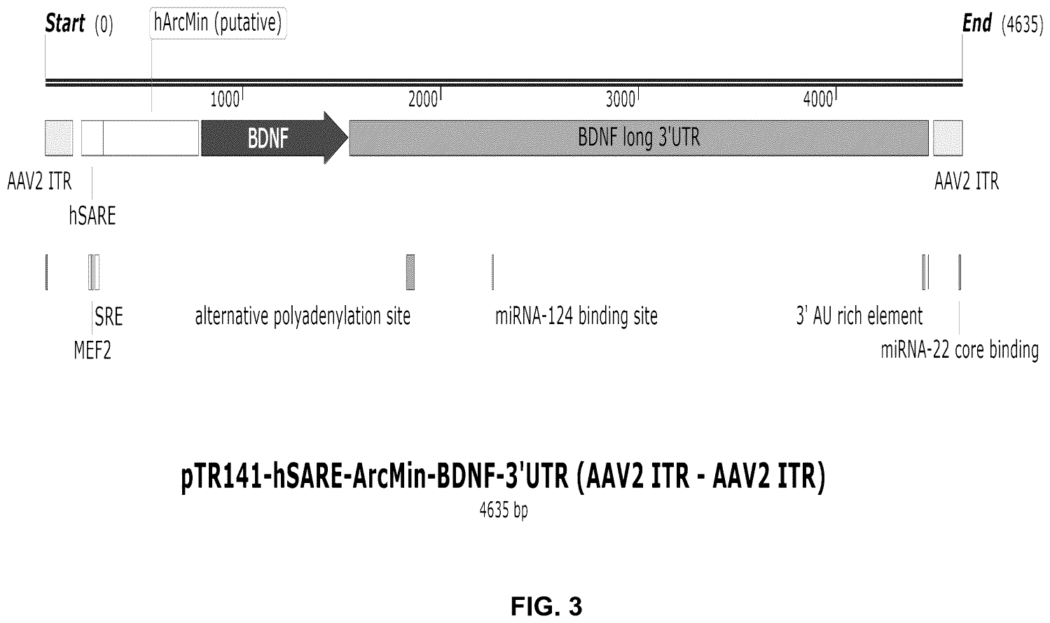

Three AAV expression cassettes comprising the activity-dependent hSARE-hArcMin promoter (SEQ ID NO: 6) and BDNF gene (SEQ ID NO: 7), flanked by 5′ ITR (SEQ ID NO: 1) and 3′ ITR (SEQ ID NO: 2), were generated using standard cloning techniques. See FIGS. 1-3 showing a schematic representation of the three cassettes. The first cassette comprises the BDNF short 3′ UTR (SEQ ID NO: 8) and a stuffer sequence (SEQ ID NO: 13). The second cassette comprises a bGH polyA signal (SEQ ID NO: 9) and a stuffer sequence (SEQ ID NO: 13). The third cassette comprises the BDNF long 3′ UTR (SEQ ID NO: 10). The three AAV expression cassettes comprise the nucleic acid sequences of SEQ ID NO: 3 (FIG. 1), SEQ ID NO: 4 (FIG. 2), and SEQ ID NO: 5 (FIG. 3), respectively.

### Example 2: Preparation of Recombinant AAV vectors in Mammalian Cells

Each of the three AAV expression cassettes is incorporated into a plasmid, to give three plasmids comprising each of the three AAV expression cassettes. Each of the three plasmids is transfected into viral production cells (e.g., HEK293) using an appropriate transfection reagent (e.g., Lipofectamine™), along with a Rep/Cap plasmid encoding the Rep and Cap genes, and a helper plasmid comprising various helper sequences required for AAV production (E4, E2a and VA). After incubation at 37° C. for a predetermined period of time, AAV particles are collected from the media or the cells are lysed to release the AAV particles. The AAV particles are then purified and titered, and may be stored at −80° C. for later use.

### Example 3: Preparation of Recombinant AAV Vectors in Insect Cells

Each of the three AAV expression cassettes are incorporated into a baculoviral vector, to give three baculoviral vectors comprising each of the three AAV expression cassettes. Insect cells (e.g., Sf9) are co-infected in suspension culture with each of the three baculoviral vectors, and least one additional recombinant baculoviral vector comprising sequences encoding the AAV Rep and Cap proteins. After incubation at 28° C. for a predetermined period of time, AAV particles are collected from the media or the cells are lysed to release the AAV particles. The AAV particles are then purified and titered, and may be stored at −80° C. for later use.

### Example 4: hSARE-hArcMin Promoter Drives Activity-Dependent Reporter Gene Expression

To test whether the hSARE-hArcMin promoter is able to drive gene expression in a manner dependent on neuronal activity, the following experiments were performed.

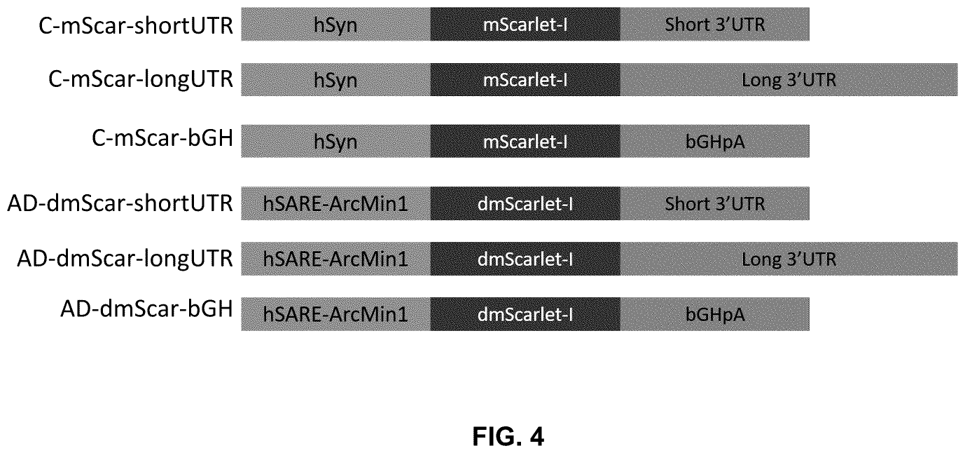

AAV expression cassettes comprising a constitutive promoter that drives long term expression in neurons (hSyn; human synapsin 1 gene promoter, comprising the nucleic acid sequence of SEQ ID NO: 38) or an activity-dependent promoter (hSARE-hArcMin, SEQ ID NO: 6) were generated using standard cloning techniques (see Table 3). FIGS. 4, 9A-9C, 10A-10C, and 11A-11C show the schematic representation of these cassettes. The cassettes comprise one of the following 3′UTR sequences: a BDNF short 3′ UTR (SEQ ID NO: 8), a bGH polyA signal (referred to as “bGHpA” or “bGH”, SEQ ID NO: 9), or a BDNF long 3′ UTR (SEQ ID NO: 10). The cassettes also comprise a reporter gene operably linked to the constitutive or activity-dependent promoter. The reporter gene encodes either a monomeric red fluorescent protein (mScarlet, encoded by the nucleic acid sequence of SEQ ID NO: 36), or a destabilized version of mScarlet (dmScarlet, encoded by the nucleic acid sequence SEQ ID NO: 35). The destabilized version of mScarlet comprises a C-terminal PEST degron signal (encoded by the nucleic acid sequence SEQ ID NO: 37), which promotes a faster turnover rate driven by proteasomal degradation, as compared to mScarlet. Therefore, dmScarlet fluorescence accumulates in the cell to a lower extent than mScarlet fluorescence.

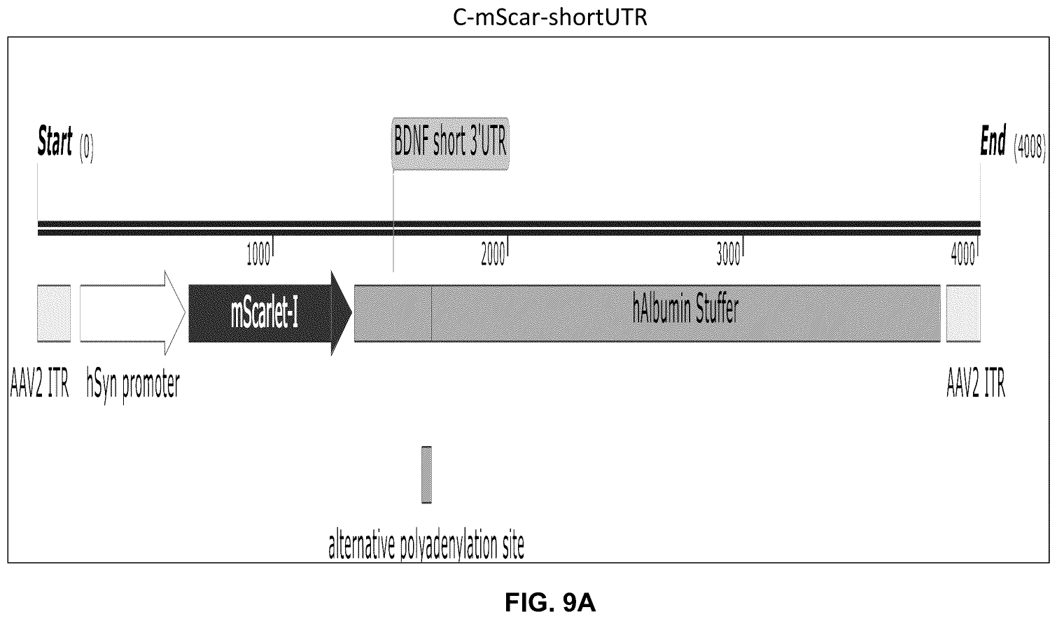

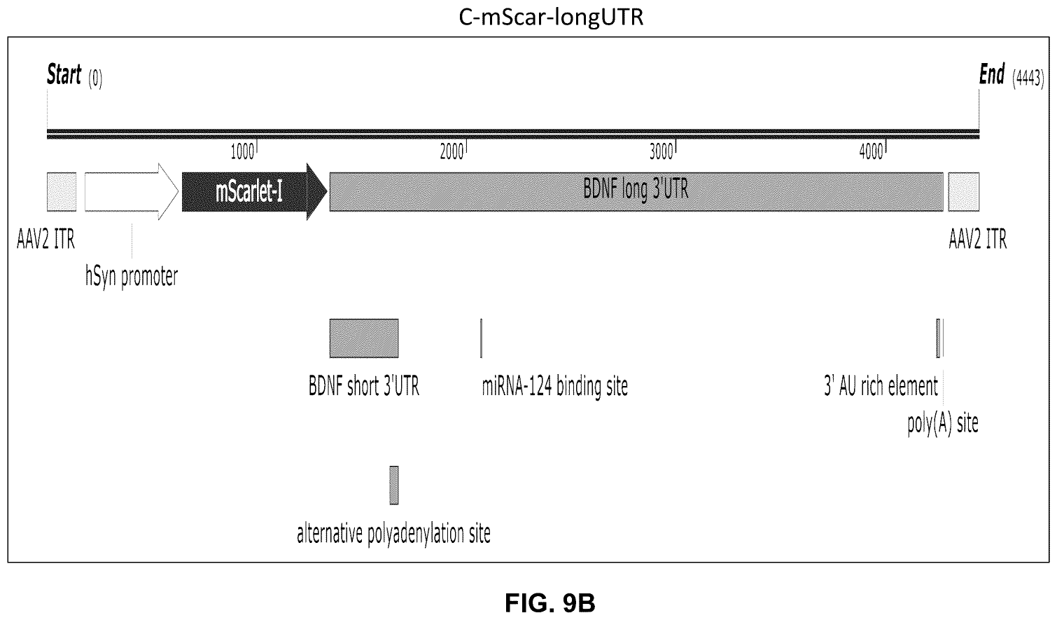

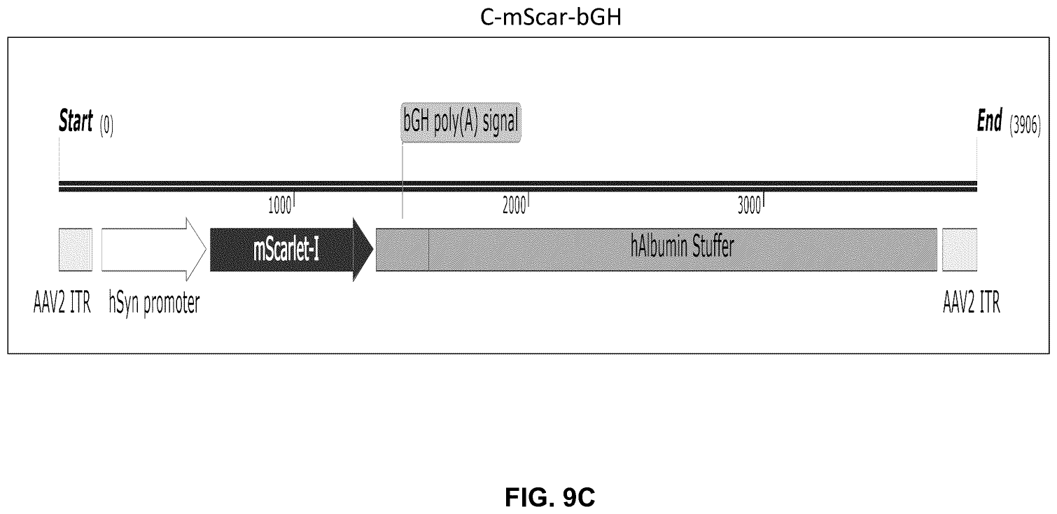

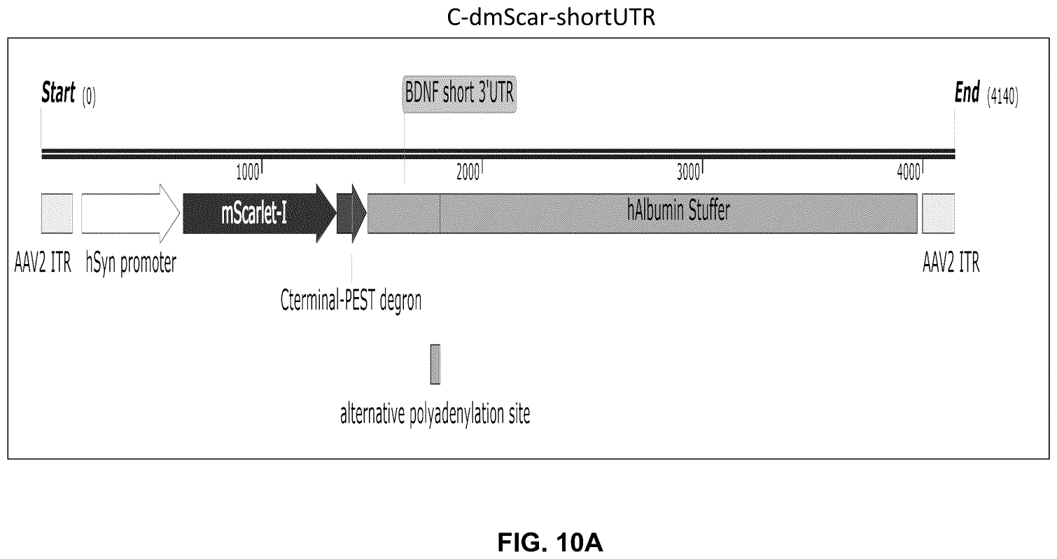

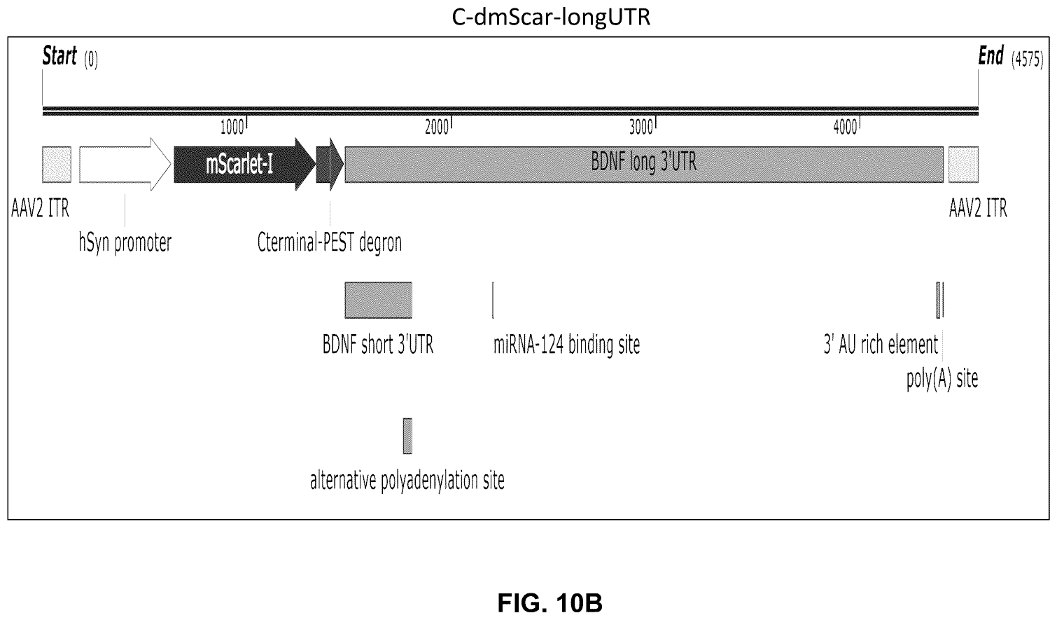

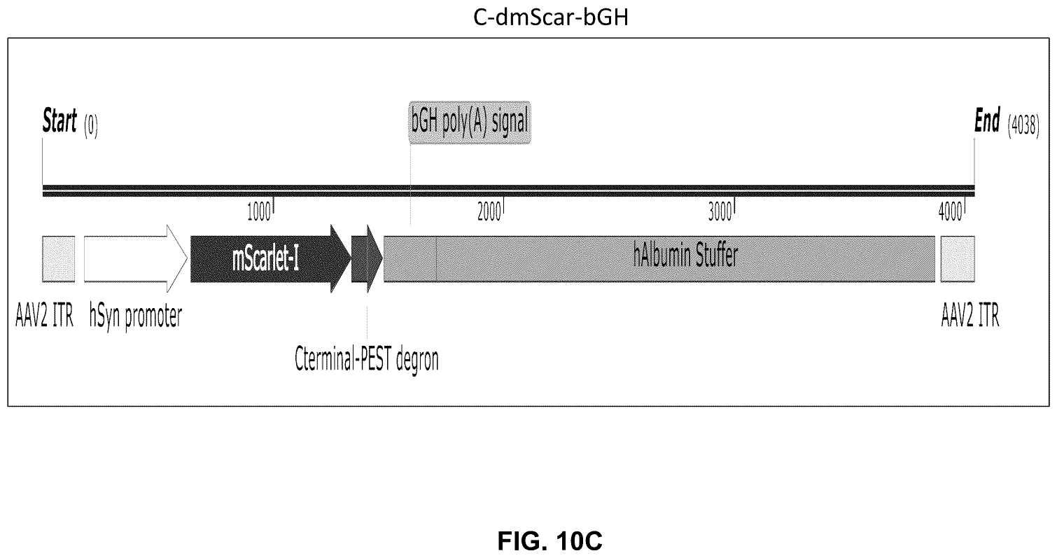

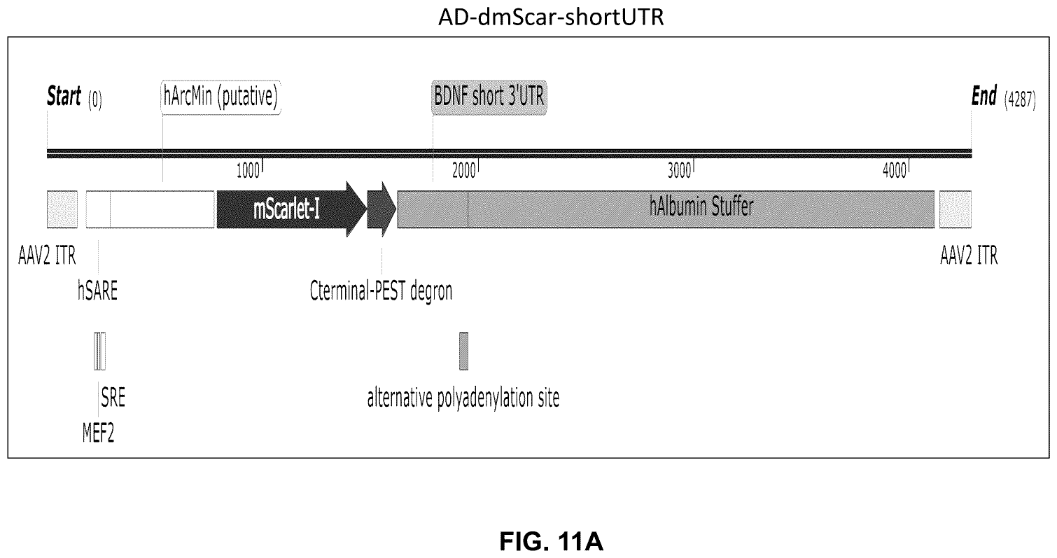

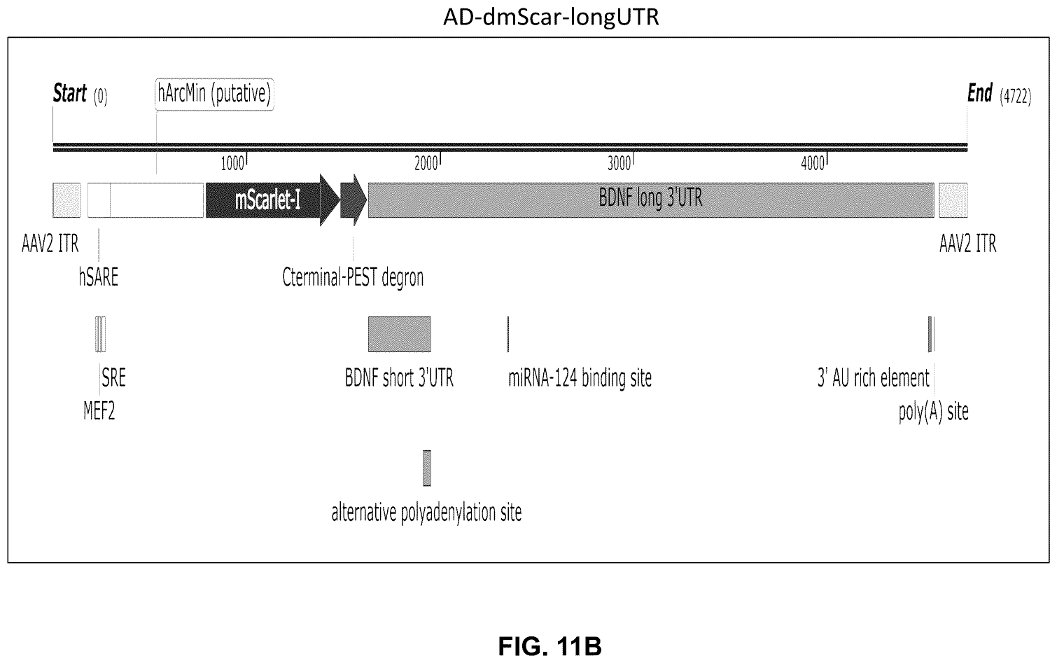

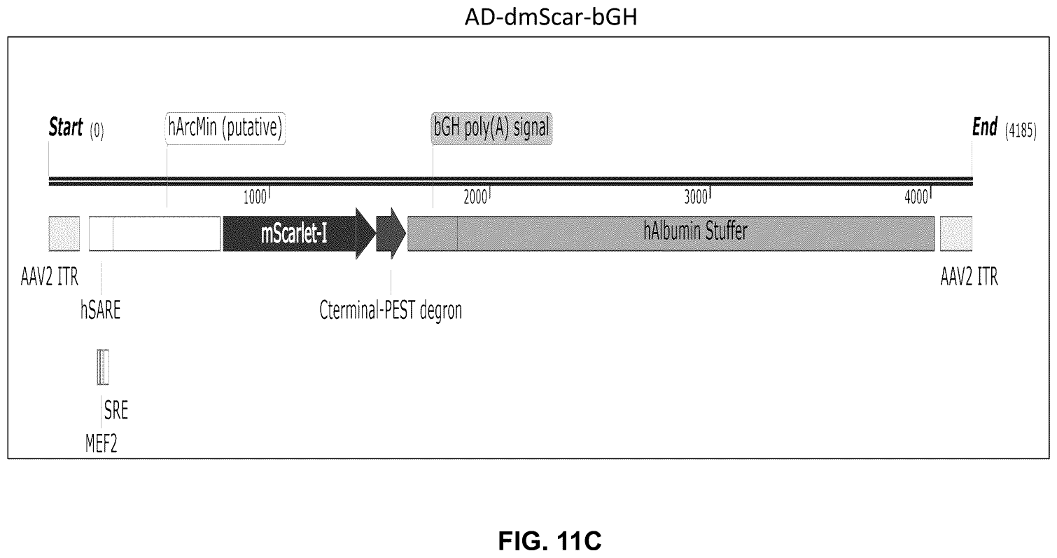

TABLE 3
Expression cassettes for reporter gene expression

| | | | | | | Sequence |
|---|---|---|---|---|---|---|
| | | SEQ | | | | downstream |
| Construct | Construct | ID | Schematic | | Reporter | of coding |
| No. | Name | NO: | representation | Promoter | Gene | region |
| 1 | C-mScar- | 26 | FIG. 9A | hSyn (Constitutive or | mScarlet-I | Short 3′UTR |
| | shortUTR | | | “C”) | (mScar) | |
| 2 | C-mScar- | 27 | FIG. 9B | hSyn (Constitutive or | mScarlet-I | Long 3′UTR |
| | longUTR | | | “C”) | (mScar) | |
| 3 | C-mScar-bGH | 28 | FIG. 9C | hSyn (Constitutive or | mScarlet-I | bGHpA |
| | | | | “C”) | (mScar) | |
| 4 | C-dmScar- | 26 | FIG. 10A | hSyn (Constitutive or | dmScarlet-I | Short 3′UTR |
| | shortUTR | | | “C”) | (dmScar) | |
| 5 | C-dmScar- | 27 | FIG. 10B | hSyn (Constitutive or | dmScarlet-I | Long 3′UTR |
| | longUTR | | | “C”) | (dmScar) | |
| 6 | C-dmScar-bGH | 28 | FIG. 10C | hSyn (Constitutive or | dmScarlet-I | bGHpA |
| | | | | “C”) | (dmScar) | |
| 7 | AD-dmScar- | 32 | FIG. 11A | hSARE-hArcMin | dmScarlet-I | Short 3′UTR |
| | shortUTR | | | (Activity-dependent | (dmScar) | |
| | | | | or “AD”) | | |
| 8 | AD-dmScar- | 33 | FIG. 11B | hSARE-hArcMin | dmScarlet-I | Long 3′UTR |
| | longUTR | | | (Activity-dependent | (dmScar) | |
| | | | | or “AD”) | | |
| 9 | AD-dmScar- | 34 | FIG. 11C | hSARE-hArcMin | dmScarlet-I | bGHpA |
| | bGH | | | (Activity-dependent | (dmScar) | |
| | | | | or “AD”) | | |

Recombinant AAV vectors comprising the AAV expression cassettes 1, 2, 3, 7, 8, and 9 listed in Table 3 were prepared as described herein. Wild type mouse primary neurons were transduced with each of these recombinant AAV vectors. The transduced cells were treated with 2 μM of the sodium channel inhibitor, tetrodotoxin (TTX) overnight. Treatment with TTX inhibits neuronal activity. Thereafter, the media was completely aspirated and replaced with any one of the following: (a) media only, (b) media+TTX, (c) media+150 mM KCl, or (d) media+30 uM bicuculine (BIC). While KCl depolarizes neurons, BIC is a competitive antagonist of the γ-Aminobutyric acid type A (GABAA) receptor and induces disinhibition of neurons. Therefore, treating neurons with either KCl or BIC promotes neuronal activity. Control neurons placed in media alone (as per treatment (a) above) are neither inhibited nor activated, and are at rest, while the activity of the neurons placed in media containing TTX (as per treatment (b) above) is inhibited. The cells were further incubated for 2 hours at 37° C. The cells were then fixed and stained with Hoescht 33342, and imaged using a fluorescence microscope.

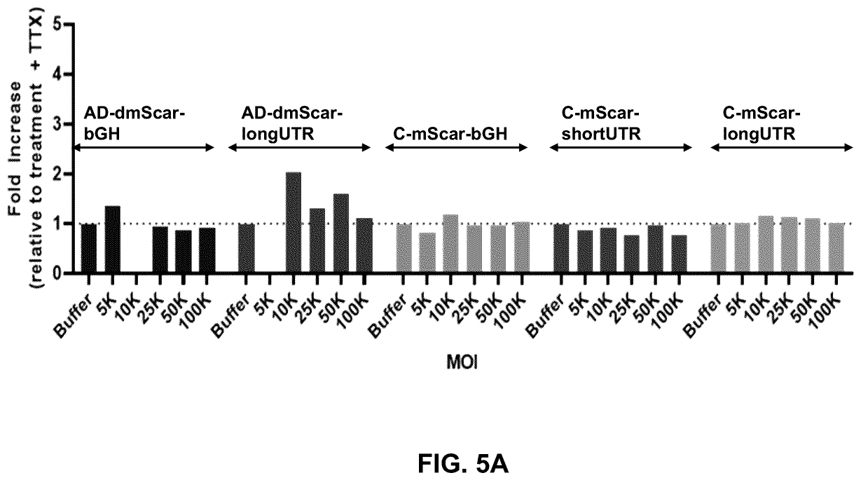

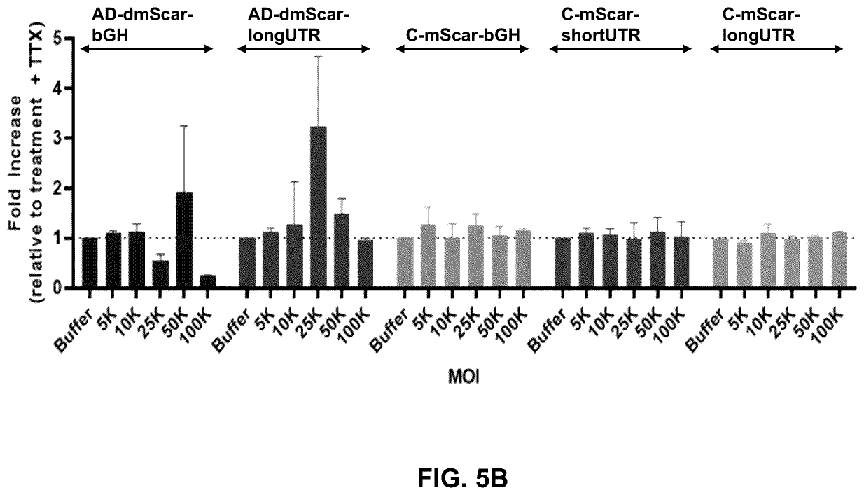

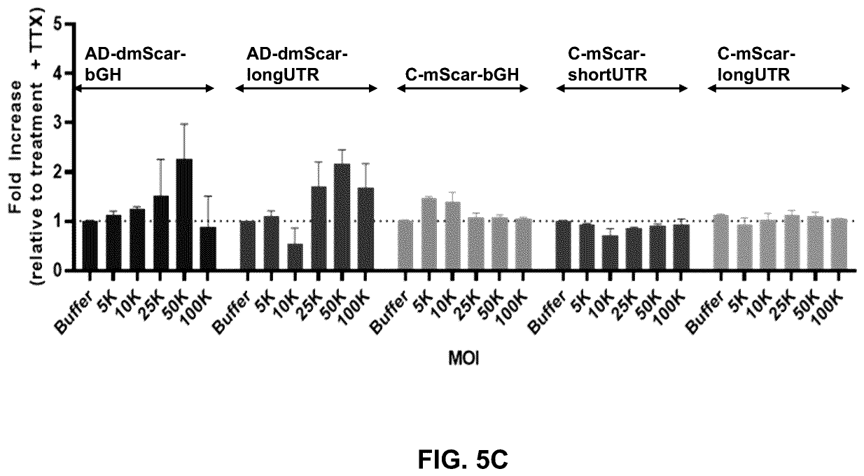

FIG. 5A shows the reporter protein fluorescence in cells that are not treated with TTX (that is, cells in which neuronal activity is neither promoted nor inhibited), relative to the fluorescence of cells that are treated with 2 μM TTX, which is normalized to 1. As shown in FIG. 5A, when neuronal activity is neither promoted nor inhibited, the relative fluorescence of cells transduced with AAV vectors that drive dmScarlet expression using the hSARE-hArcMin promoter is comparable to the relative fluorescence of cells transduced with AAV vectors that drive mScarlet expression using the constitutive promoter, hSyn.

FIG. 5B shows the reporter protein fluorescence in cells that are not treated with TTX but instead treated with 150 mM KCl (that is, cells in which neuronal activity is promoted), relative to the fluorescence of cells that are treated with 2 μM TTX, which is normalized to 1. FIG. 5C shows the reporter protein fluorescence in cells that are not treated with TTX but instead treated with 30 uM bicuculine (BIC) (that is, cells in which neuronal activity is promoted), relative to the fluorescence of cells that are treated with 2 μM TTX, which is normalized to 1.

As shown in FIGS. 5B and 5C, when neuronal activity is promoted using KCl or BIC treatment, the relative fluorescence of cells transduced with AAV vectors that drive dmScarlet expression using the activity-dependent promoter, hSARE-hArcMin is higher than the relative fluorescence of cells transduced with AAV vectors that drive mScarlet expression using the constitutive promoter (hSyn).

These results demonstrate that the hSARE-hArcMin promoter is able to drive gene expression in a manner dependent on neuronal activity. Furthermore, dmScarlet constructs comprising the bGH or the long 3′UTR were similarly expressed by the hSARE-hArcMin promoter, as shown in FIGS. 5B and 5C.

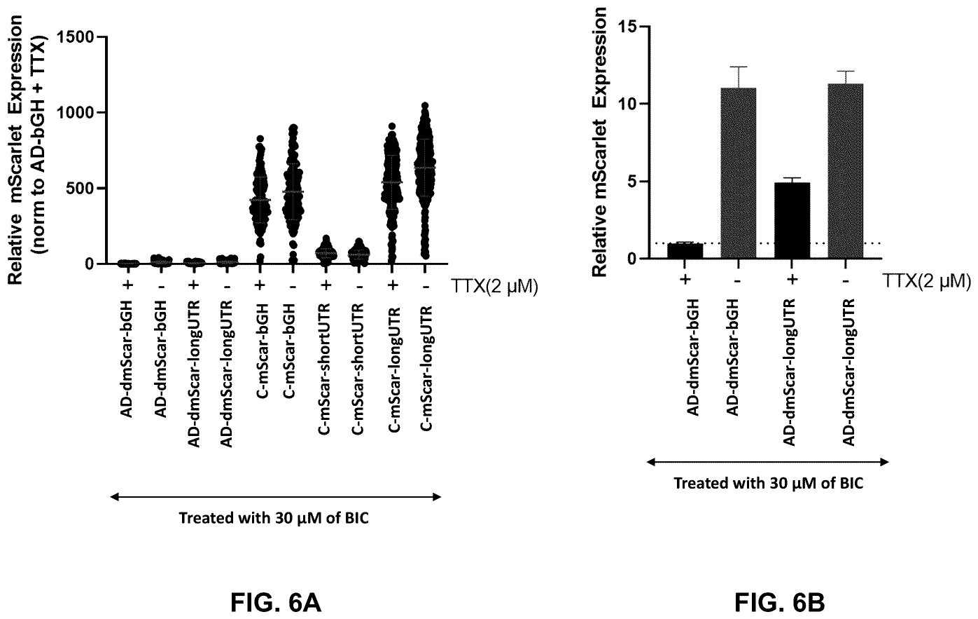

The activity-dependent expression of the hSARE-hArcMin promoter is further illustrated by FIG. 6A-B. As shown in FIG. 6A, the constitutive promoter, hSyn, drives high levels of mScarlet expression, irrespective of the presence of TTX. Strikingly, FIG. 6B shows that when cells are not treated with TTX, but instead treated with 30 uM of BIC to promote neuronal activity, the hSARE-hArcMin promoter drives high levels of reporter gene expression. Notably, FIG. 6B shows that the AAV expression construct comprising the hSARE-hArcMin promoter in combination with long 3′UTR resulted in about 5-fold higher level of dmScarlet expression in the presence of TTX, as compared to the AAV expression construct comprising the hSARE-hArcMin promoter in combination bGHpA. These results further demonstrates the neuronal activity-dependent induction of the hSARE-hArcMin promoter.

To determine the subcellular localization and levels of the expressed fluorescent protein, neurons expressing the reporter gene under the constitutive hSyn promoter or the activity-dependent hSARE-hArcMin promoter were observed using microscopy.

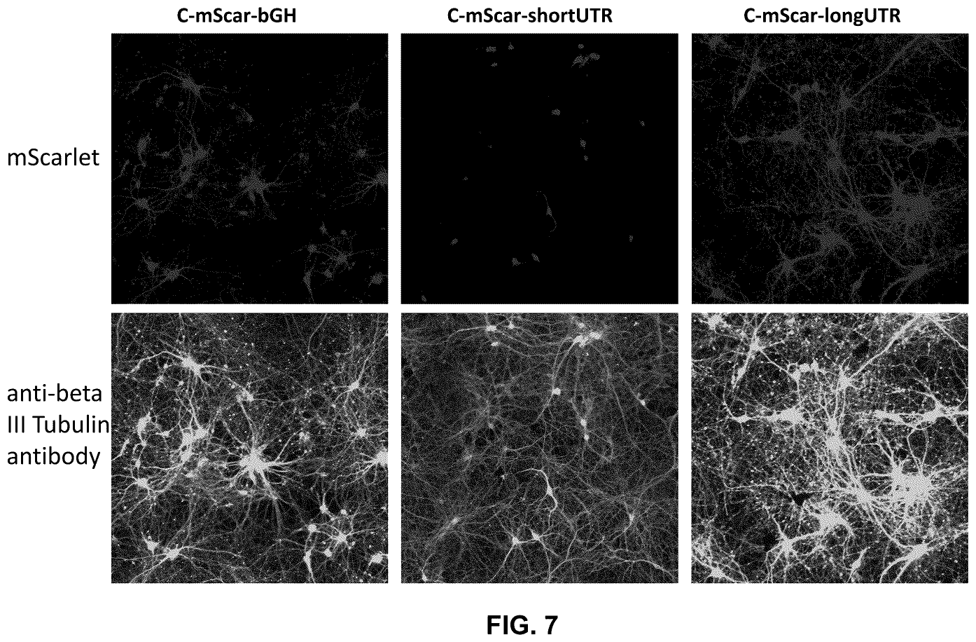

As shown in FIG. 7, cells expressing mScarlet using the constitutive promoter, hSyn, were fluorescent, however, the level of fluorescence varied based on the 3′UTR region or polyadenylation signal of the expression cassette. For instance, expression cassettes comprising the long 3′UTR resulted in the higher levels of mScarlet gene expression, followed by the expression cassettes comprising the short 3′UTR. Expression cassettes comprising the short 3′UTR resulted in the considerably lower levels of mScarlet gene expression. In contrast, the expression of the reporter protein under the activity-dependent hSARE-hArcMin promoter do not vary based on the type of 3′UTR used. In particular, FIGS. 5 and 6 show that AAV expression constructs comprising the hSARE-hArcMin promoter in combination with bGHpA or long 3′UTR resulted in similar levels of dmScarlet expression in the presence of neuronal activity.

Furthermore, FIG. 7 shows that the subcellular localization of mScarlet expressed from a constitutive promoter varies based on the 3′UTR region or polyadenylation signal of the expression cassette. While expression cassettes comprising the long 3′UTR and bGHpA resulted in mScarlet expression in the cell body and neurites, expression cassettes comprising the short 3′UTR resulted in mScarlet expression only in the cell body.

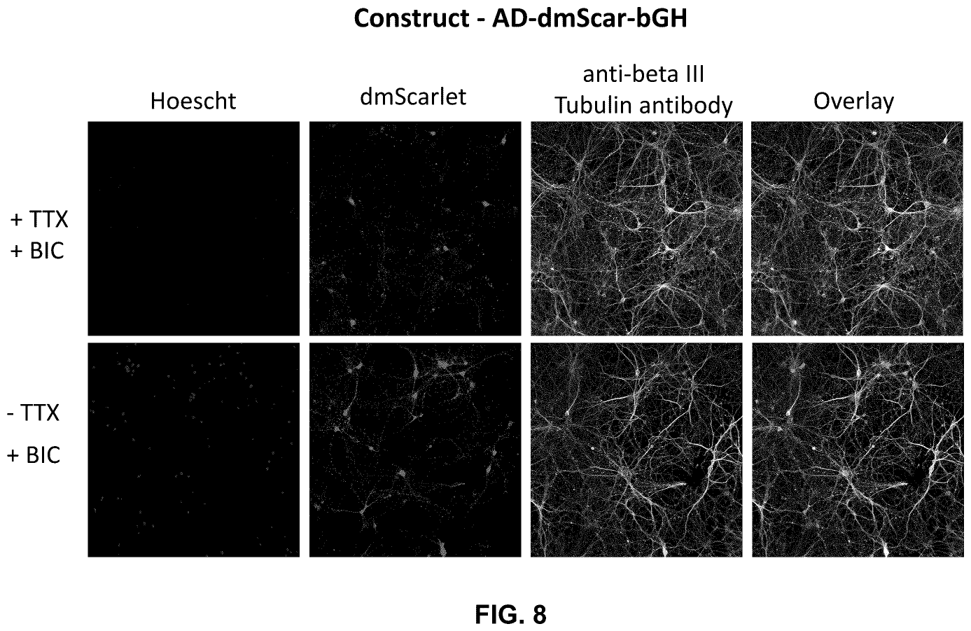

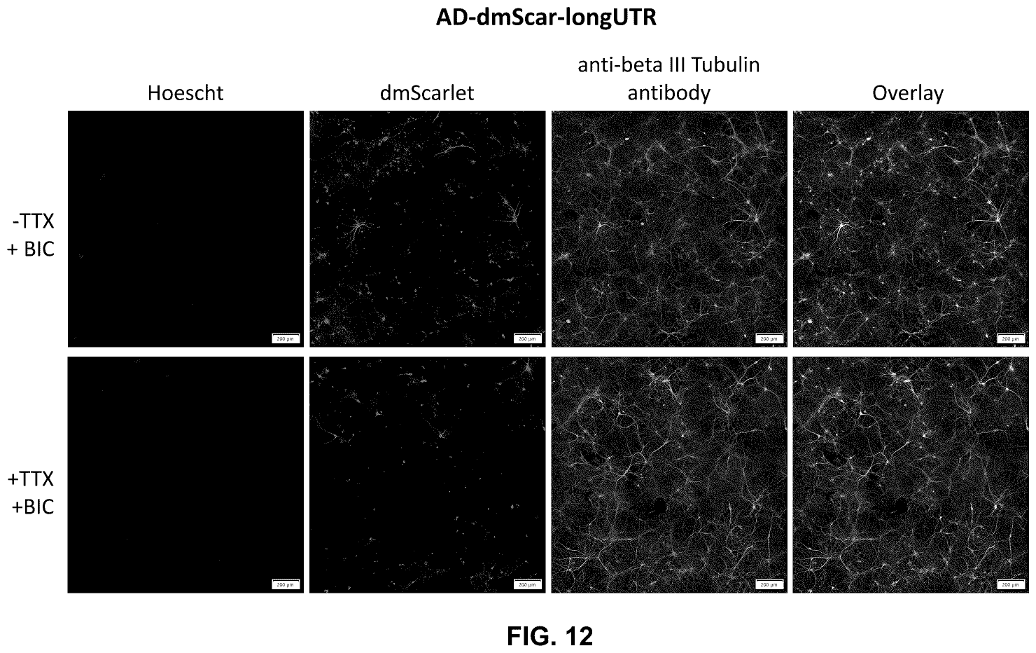

As shown in FIGS. 8 and 12, AAV expression constructs comprising the hSARE-hArcMin promoter in combination with bGHpA (FIG. 8) or long 3′UTR (FIG. 12) resulted in similar levels of dmScarlet expression in the presence of neuronal activity. Furthermore, cells expressing dmScarlet using the activity-dependent hSARE-hArcMin promoter showed fluorescence signal in the cell body and neurites upon neuronal activation (in the absence of TTX and presence of BIC).

In sum, the results described above demonstrate that the synthetic hSARE-hArcMin promoter promotes higher levels of gene expression in neurons that are active, as compared to in neurons that are inhibited or resting. Therefore, the synthetic hSARE-hArcMin promoter may be used to drive neuronal activity-dependent expression of any target gene, such as, a Rett Syndrome-associated gene as described herein.

### Example 5: Characterization of AAV Expression Cassettes Comprising BDNF

The AAV particles generated in Example 2 or 3 are administered by injection into mecp2 mutant mice, which exhibit symptoms similar to Rett Syndrome, to test whether the expression of BDNF using the activity dependent promoter, hSARE-hArcMin, decreases the severity of one or more symptoms. The ability of activity dependent BDNF expression to delay the onset of Rett syndrome symptoms in newborn mecp2 mutant mice is also tested.

The foregoing is illustrative of the present invention, and is not to be construed as limiting thereof.

### NUMBERED EMBODIMENTS

The following list of embodiments is included herein for illustration purposes only and is not intended to be comprehensive or limiting. The subject matter to be claimed is expressly not limited to the following embodiments.

Embodiment 1. A nucleic acid comprising an adeno-associated virus (AAV) expression cassette, wherein the AAV expression cassette comprises, from 5′ to 3′:
a 5′ inverted terminal repeat (ITR);
a synthetic, activity-dependent promoter;
a Rett Syndrome-associated gene; and
a 3′ ITR.

Embodiment 2. The nucleic acid of embodiment 1, wherein the promoter drives expression of the Rett-syndrome-associated gene.

Embodiment 3. The nucleic acid of embodiment 1 or 2, wherein the promoter is a MECP2-independent promoter.

Embodiment 4. The nucleic acid of any one of embodiments 1-3, wherein the promoter comprises a nucleic acid sequence derived from a promoter of a neuronal immediate early gene.

Embodiment 5. The nucleic acid of embodiment 4, wherein the neuronal immediate early gene is selected from the group consisting of the Arc gene, the c-fos gene, and the egr-1 gene.

Embodiment 6. The nucleic acid of any one of embodiments 1-5, wherein the promoter comprises a minimal Arc gene promoter (ArcMin).

Embodiment 7. The nucleic acid of embodiment 6, wherein the ArcMin is a human ArcMin (hArcMin).

Embodiment 8. The nucleic acid of embodiment 7, wherein the hArcMin comprises a nucleic acid sequence of SEQ ID NO: 12, or a sequence at least 90% identical thereto.

Embodiment 9. The nucleic acid of any one of embodiments 1-8, wherein the promoter comprises a cyclic AMP response element (CRE), a serum response element (SRE), a synaptic activity response element (SARE), a MEF2 response element, or a combination thereof.

Embodiment 10. The nucleic acid of embodiment 9, wherein the promoter comprises a synaptic activity response element (SARE).

Embodiment 11. The nucleic acid of embodiment 10, wherein the synaptic activity response element (SARE) is a human synaptic activity response element (hSARE).

Embodiment 12. The nucleic acid of embodiment 11, wherein the hSARE comprises a nucleic acid sequence of SEQ ID NO: 11, or a sequence at least 90% identical thereto.

Embodiment 13. The nucleic acid of any one of embodiments 1-12, wherein the promoter comprises a human ArcMin (hArcMin) and at least one hSARE.

Embodiment 14. The nucleic acid of embodiment 13, wherein the promoter comprises hArcMin and one hSARE.

Embodiment 15. The nucleic acid of embodiment 14, wherein the promoter comprises a nucleic acid sequence of SEQ ID NO: 6, or a sequence at least 90% identical thereto. Embodiment 16. The nucleic acid of embodiment 13, wherein the promoter comprises hArcMin and five hSAREs

Embodiment 17. The nucleic acid of embodiment 16, wherein the promoter comprises a nucleic acid sequence of SEQ ID NO: 16, or a sequence at least 90% identical thereto. Embodiment 18. The nucleic acid of any one of embodiments 1-17, wherein the promoter binds to a neuronal activity dependent transcription factor.

Embodiment 19. The nucleic acid of embodiment 18, wherein the neuronal activity dependent transcription factor is cAMP-responsive element binding protein (CREB), myocyte enhancer factor 2 (MEF2), serum response factor (SRF), or Elk-1.

Embodiment 20. The nucleic acid of any one of embodiments 1-19, wherein the Rett-syndrome-associated gene encodes brain-derived neurotrophic factor (BDNF), insulin-like growth factor 1 (IGF1), methyl-CpG-binding protein 2 (MECP2), Huntingtin protein, Huntington-associated protein 1, Orthodenticle homeobox 2 (OTX-2), FXYD Domain Containing Ion Transport Regulator 1 (FXYD1), Neurexin-2-alpha (NRXN2), or Protein Kinase C Gamma (PRKCG).

Embodiment 21. The nucleic acid of embodiment 20, wherein the Rett-syndrome-associated gene encodes brain-derived neurotrophic factor (BDNF).

Embodiment 22. The nucleic acid of embodiment 21, wherein the BDNF is a human BDNF.

Embodiment 23. The nucleic acid of embodiment 22, wherein the BDNF is encoded by a nucleic acid sequence of SEQ ID NO: 7, or a sequence at least 90% identical thereto. Embodiment 24. The nucleic acid of any one of embodiments 1-23, wherein at least one of the 5′ ITR and the 3′ ITR is about 110 to about 160 nucleotides in length.

Embodiment 25. The nucleic acid of any one of embodiments 1-24, wherein the 5′ ITR is the same length as the 3′ ITR.

Embodiment 26. The nucleic acid of any one of embodiments 1-24, wherein the 5′ ITR and the 3′ ITR have different lengths.

Embodiment 27. The nucleic acid of any one of embodiments 1-26, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

Embodiment 28. The nucleic acid of any one of embodiments 1-23, wherein the 5′ ITR comprises the sequence of SEQ ID NO: 1.

Embodiment 29. The nucleic acid of any one of embodiments 1-23, wherein the 3′ ITR comprises the sequence of SEQ ID NO: 2.

Embodiment 30. The nucleic acid of any one of embodiments 1-29, wherein the AAV cassette comprises a brain-derived neurotrophic factor (BDNF) short 3′ UTR, or a BDNF long 3′ UTR.

Embodiment 31. The nucleic acid of embodiment 30, wherein the BDNF short 3′ UTR, or the BDNF long 3′ UTR is located between the Rett-syndrome-associated gene and the 3′ ITR.

Embodiment 32. The nucleic acid of embodiment 30 or embodiment 31, wherein the AAV cassette comprises a BDNF short 3′ UTR.

Embodiment 33. The nucleic acid of embodiment 32, wherein the BDNF short 3′ UTR comprises a nucleic acid sequence of SEQ ID NO: 8, or a sequence at least 90% identical thereto.

Embodiment 34. The nucleic acid of embodiment 30 or embodiment 31, wherein the AAV cassette comprises a BDNF long 3′ UTR.

Embodiment 35. The nucleic acid of embodiment 34, wherein the BDNF long 3′ UTR comprises a nucleic acid sequence of SEQ ID NO: 10, or a sequence at least 90% identical thereto.

Embodiment 36. The nucleic acid of any one of embodiments 1-35, wherein the AAV cassette comprises a polyadenylation signal.

Embodiment 37. The nucleic acid of embodiment 36, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).

Embodiment 38. The nucleic acid of embodiment 36, wherein the AAV cassette comprises a bGH polyadenylation signal.

Embodiment 39. The nucleic acid of embodiment 38, wherein the bGH polyadenylation signal comprises a nucleic acid sequence of SEQ ID NO: 9, or a sequence at least 90% identical thereto.

Embodiment 40. The nucleic acid of any one of embodiments 1-39, wherein the AAV cassette comprises at least one stuffer sequence.

Embodiment 41. The nucleic acid of embodiment 40, wherein the at least one stuffer sequence comprises a nucleic acid sequence of SEQ ID NO: 13, or a sequence at least 90% identical thereto.

Embodiment 42. The nucleic acid of any one of embodiments 1-41, wherein the AAV expression cassette comprises a Kozak sequence, wherein the Kozak sequence overlaps with the start codon of the Rett-syndrome-associated gene.

Embodiment 43. The nucleic acid of embodiment 42, wherein the Kozak sequence comprises a nucleic acid sequence of SEQ ID NO: 14, or a sequence at least 90% identical thereto; or a nucleic acid sequence of SEQ ID NO: 15 (acagccacc), or a sequence at least 90% identical thereto.

Embodiment 44. The nucleic acid of any one of embodiments 1-43, wherein the AAV expression cassette comprises a nucleic acid sequence SEQ ID NO: 3, or a sequence at least 90% identical thereto; SEQ ID NO: 4 or a sequence at least 90% identical thereto; or SEQ ID NO: 5 or a sequence at least 90% identical thereto.

Embodiment 45. A plasmid comprising the nucleic acid of any one of embodiments 1-44.

Embodiment 46. A cell comprising the nucleic acid of any one of embodiments 1-44 or the plasmid of embodiment 45.

Embodiment 47. A method of producing a recombinant AAV vector, the method comprising contacting an AAV producer cell with the nucleic acid of any one of embodiments 1-44, or the plasmid of embodiment 45.

Embodiment 48. A recombinant AAV vector produced by the method of embodiment 47.

Embodiment 49. The recombinant AAV vector of embodiment 48, wherein the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.

Embodiment 50. The recombinant AAV vector of embodiment 48 or embodiment 49, wherein the recombinant AAV vector is a single-stranded AAV (ssAAV).

Embodiment 51. The recombinant AAV vector of any one of embodiments 48-50, wherein the recombinant AAV vector is a self-complementary AAV (scAAV).

Embodiment 52. The recombinant AAV vector of any one of embodiments 48-51, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10,AAVrh32.33, AAVrh74, Avian AAV or Bovine AAV.

Embodiment 53. The recombinant AAV vector of any one of embodiments 48-52, wherein the AAV vector comprises a capsid protein with one or more substitutions or mutations, as compared to a wildtype AAV capsid protein.

Embodiment 54. A composition, comprising: (a) the nucleic acid of any one of embodiments 1-44, the plasmid of embodiment 45, the cell of embodiment 46, or the recombinant AAV vector of any one of embodiments 48-53; and (b) a pharmaceutically acceptable carrier.

Embodiment 55. A method for expressing a Rett-syndrome-associated gene in a subject in need thereof, comprising administering to the subject a therapeutically effective amount of the nucleic acid of any one of embodiments 1-44, the plasmid of embodiment 45, the cell of embodiment 46, the recombinant AAV vector of any one of embodiments 48-53, or the composition of embodiment 54.

Embodiment 56. The method of embodiment 55, wherein the subject has Rett syndrome.

Embodiment 57. A method for treating or delaying the onset of Rett syndrome in a subject, comprising administering to the subject a therapeutically effective amount of the nucleic acid of any one of embodiments 1-44, the plasmid of embodiment 45, the cell of embodiment 46, the recombinant AAV vector of any one of embodiments 48-53, or the composition of embodiment 54.

Embodiment 58. A method for expressing brain-derived neurotrophic factor (BDNF) in a subject in need thereof, comprising administering to the subject a therapeutically effective amount of the nucleic acid of any one of embodiments 1-44, the plasmid of embodiment 45, the cell of embodiment 46, the recombinant AAV vector of any one of embodiments 48-53, or the composition of embodiment 54.

Embodiment 59. The method of embodiment 58, wherein the subject has a cognitive disorder, or a stress-related disorder.

Embodiment 60. The method of embodiment 58, wherein the subject has depression, obsessive-compulsive disorder, Alzheimer's disease, Huntington's disease, and dementia, anorexia nervosa and bulimia nervosa, schizophrenia, epilepsy, post-traumatic stress disorder, obesity, Rett syndrome, or post-chemotherapy cognitive impairment.

Embodiment 61. A method of treating or delaying the onset of a BDNF-associated disease in a subject, comprising administering to the subject a therapeutically effective amount of the nucleic acid of any one of embodiments 1-44, the plasmid of embodiment 45, the cell of embodiment 46, the recombinant AAV vector of any one of embodiments 48-53, or the composition of embodiment 54.

Embodiment 62. The method of embodiment 61, wherein the BDNF-associated disease is a cognitive disorder, and/or a stress-related disorder.

Embodiment 63. The method of embodiment 61, wherein the BDNF-associated disease is depression, obsessive-compulsive disorder, Alzheimer's disease, Huntington's disease, and dementia, anorexia nervosa, bulimia nervosa, schizophrenia, epilepsy, post-traumatic stress disorder, bipolar disease, Rett syndrome, major depressive disorder, or post-chemotherapy cognitive impairment.

Embodiment 64. A method of treating or delaying the onset of a MECP2-associated disease in a subject, comprising administering to the subject a therapeutically effective amount of the nucleic acid of any one of embodiments 1-44, the plasmid of embodiment 45, the cell of embodiment 46, the recombinant AAV vector of any one of embodiments 48-53, or the composition of embodiment 54.

Embodiment 65. The method of embodiment 64, wherein the MECP2-associated disease is MECP2 duplication syndrome, MECP2-related severe neonatal encephalopathy, PPM-X syndrome, or Rett syndrome.

Embodiment 66. The method of any one of embodiment 55-65, wherein the subject is a human subject.

Embodiment 67. The method of any one of embodiments 55-66, wherein the nucleic acid, the plasmid, the cell, the recombinant AAV vector, or composition is administered by injection into the central nervous system.

Embodiment 68. The method of any one of embodiments 55-67, wherein the Rett Syndrome-associated gene is expressed in the neurons of the subject.

Embodiment 69. The method of embodiment 68, wherein the neurons are active neurons.

1-32. (canceled)

33. A method of treating or delaying the onset of a MECP2-associated disease in a subject, comprising administering to the subject a therapeutically effective amount of a nucleic acid comprising an adeno-associated virus (AAV) expression cassette, wherein the AAV expression cassette comprises, from 5′ to 3′:
a 5′ inverted terminal repeat (ITR);
a minimal human Arc gene promoter (hArcMin) set forth by SEQ ID NO: 12;
a Rett Syndrome-associated gene; and
a 3′ ITR.

34. The method of claim 33, wherein the minimal human Arc gene promoter (hArcMin) drives expression of the Rett-syndrome-associated gene.

35. The method of claim 34, wherein the nucleic acid further comprises a cyclic AMP response element (CRE), a serum response element (SRE), a synaptic activity response element (SARE), a myocyte enhancer factor 2 (MEF2) response element, or a combination thereof.

36. The method of claim 35, wherein the nucleic acid comprises at least one synaptic activity response element (SARE).

37. The method of claim 36, wherein the synaptic activity response element (SARE) is a human synaptic activity response element (hSARE) of SEQ ID NO: 11, or a sequence at least 95% identical thereto.

38. The method of claim 37, wherein the hArcMin and SARE are set forth by SEQ ID NO: 6, or a sequence at least 95% identical thereto.

39. The method of claim 37, wherein the hArcMin and SARE are set forth by SEQ ID NO: 16, or a sequence at least 95% identical thereto.

40. The method of claim 33, wherein the Rett-syndrome-associated gene encodes brain-derived neurotrophic factor (BDNF), insulin-like growth factor 1 (IGF1), methyl-CpG-binding protein 2 (MECP2), Huntingtin protein, Huntington-associated protein 1, Orthodenticle homeobox 2 (OTX-2), FXYD Domain Containing Ion Transport Regulator 1 (FXYD1), Neurexin-2-alpha (NRXN2), or Protein Kinase C Gamma (PRKCG).

41. The method of claim 33, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV, or Bovine AAV.

42. The method of claim 34, wherein the 5′ ITR comprises the sequence of SEQ ID NO: 1, or the 3′ ITR comprises the sequence of SEQ ID NO: 2, or a combination thereof.

43. The method of claim 34, wherein the AAV cassette comprises one of the following located between the Rett-syndrome-associated gene and the 3′ ITR: (a) a brain-derived neurotrophic factor (BDNF) short 3′ UTR, (b) a BDNF long 3′ UTR, or (c) a polyadenylation signal.

44. The method of claim 43, wherein the AAV cassette comprises a BDNF short 3′ UTR of SEQ ID NO: 8.

45. The method of claim 43, wherein the AAV cassette comprises a BDNF long 3′ UTR of SEQ ID NO: 10.

46. The method of claim 43, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).

47. The method of claim 46, wherein the AAV cassette comprises a bGH polyadenylation signal and wherein the bGH polyadenylation signal comprises a nucleic acid sequence of SEQ ID NO: 9, or a sequence at least 90% identical thereto.

48. The method of claim 34, wherein the AAV expression cassette comprises one of the following:
(a) a nucleic acid sequence of SEQ ID NO: 3, or a sequence at least 95% identical thereto; (b) a nucleic acid sequence of SEQ ID NO: 4 or a sequence at least 95% identical thereto; or (c) a nucleic acid sequence of SEQ ID NO: 5 or a sequence at least 95% identical thereto.

49. The method of claim 34, wherein the MECP2-associated disease is MECP2 duplication syndrome, MECP2-related severe neonatal encephalopathy, PPM-X syndrome, or Rett syndrome.

50. The method of claim 34, wherein the Rett Syndrome-associated gene is expressed in the neurons of the subject.

51. The method of claim 50, wherein the neurons are active neurons.
